(19) United States
(12) Patent Application Publication
(10) Pub. No.: US 2023/0399378 A1
(43) Pub. Date: Dec. 14, 2023

(54) PEPTIDE-BASED DELIVERY OF AGENTS

(71) Applicant: THE FLOREY INSTITUTE OF NEUROSCIENCE AND MENTAL HEALTH, Parkville (AU)

(72) Inventors: Fazel SHABANPOOR, Parkville (AU); Bradley TURNER, Parkville (AU)

(21) Appl. No.: 18/034,526

(22) PCT Filed: Oct. 28, 2021
(86) PCT No.: PCT/AU2021/051255

§ 371 Date: Apr. 28, 2023

(30) Foreign Application Priority Data
Oct. 28, 2020 (AU) 2020903909

(51) Int. Cl. C07K 14/775; C12N 15/113; A61K 47/64

(52) U.S. Cl. CPC C07K 14/775; C12N 15/113; A61K 47/64; C07K 2319/01; C07K 2319/09; C12N 2310/3513; C12N 2310/314; C12N 2320/32; C12N 2320/33

(57) ABSTRACT

Described herein are fusion peptides comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain. Such fusion peptides can be conjugated with an active agent to deliver the active agent across the blood-brain barrier to the central nervous system and across blood-ocular barriers to the eye.

### FIELD OF THE ART

The present disclosure relates to peptides, in particular fusion peptides, capable of crossing the blood-brain barrier and blood-ocular barriers, and more particularly to the use of such peptides and fusion peptides as shuttles to facilitate the delivery of active agents, such as antisense oligonucleotides, across the blood-brain barrier to the central nervous system and the delivery of active agents, such as antisense oligonucleotides, across blood-ocular barriers to the eye.

### BACKGROUND

The blood-brain barrier presents a semi-permeable barrier between the circulatory system and the central nervous system (CNS), by which the microvasculature of the CNS tightly regulates the movement of molecules, ions, and cells between the blood and the CNS. This allows endothelial cells of the blood-brain barrier to regulate CNS homeostasis, which is critical for proper neuronal function, as well as to protect the CNS from toxins, pathogens, inflammation, injury, and disease. Similarly, the blood-aqueous humor barrier (also known as the blood-aqueous barrier) and the blood-retinal barrier (together, the blood-ocular barriers) present a physical semi-permeable barrier between intraocular tissues and the surrounding blood vessels.

Neuromuscular and neurodegenerative conditions affecting the CNS remain the world's leading cause of disability and account for more hospitalizations and need for prolonged care than almost all other diseases combined. The personal effects of these CNS disorders on sufferers and their families, as well as on society as a whole, are profound.

Despite advances in the identification of drugs capable of treating CNS and ocular disorders, the restrictive nature of the blood-brain barrier and the blood-ocular barriers present a significant obstacle for drug delivery. For example, the majority of drugs developed for the treatment of neurological and neuromuscular disorders are limited in efficacy by their poor penetration of the blood-brain barrier. Thus, despite significant progress in the development of novel therapeutics for the treatment of diseases and disorders affecting the CNS offering great promise for sufferers, including antisense oligonucleotides, peptides and small molecules, there remains a challenge in effectively delivering these therapeutics to their site of action in the CNS, particularly in the brain, in sufficient amounts and with sufficient bioavailability to be clinically effective. In the case of ophthalmic treatments, many drugs are formulated as solutions, suspensions or ointments for topical administration since insufficient intraocular drug concentrations can be achieved via oral and intravenous routes.

Direct injection of drugs into the brain or spinal column, thus bypassing the blood-brain barrier is not only highly invasive and expensive, but is ineffective due to the small volume of drug that can be administered and poor diffusion of the drug from the injection site. Disrupting the blood-brain barrier, for example, by infusing hyperosmolar solutions to remove water from capillary endothelial cells, thereby shrinking them to open gaps, or the use of bradykinin receptor agonists to loosen tight junctions, is also of limited effectiveness and suffers from the drawback that blood-brain barrier disruption weakens the barrier and allows the non-specific entry of other, potentially toxic molecules.

Numerous antisense oligonucleotide-based therapeutics are in clinical trial, including several for the treatment of neurological conditions. One hurdle associated with bringing these therapeutics to the clinic is the lack of non-invasive and efficient delivery vehicles. Antisense oligonucleotides targeting the CNS are typically delivered through multiple invasive intrathecal injections at higher doses than are needed for therapeutic effect, which have been reported to have significant side-effects. Nusinersen is a 20-mer antisense oligonucleotide designed to target and correct splicing of the SMN2 gene for the treatment of spinal muscular atrophy. Nusinersen is susceptible to nucleases and is incapable of crossing the blood-brain barrier. As such, it is administered via multiple invasive intrathecal injections with limited beneficial effect and significant side effects; for example Nusinersen injection has been reported to show side effects such as postprocedural headache and backpain in 32% of adult patients.

Thus, significant efforts have been made to generate methods to improve the ability of drugs to penetrate the blood-brain barrier and the blood-ocular barriers, including the use of molecules such as peptides to ‘shuttle’ or carry drugs across these barriers. Peptides offer significant advantages as a vehicle for the delivery of agents across the blood-brain barrier, including small size, low toxicity, targeting specificity and their ability to carry large molecular weight agents. However, to date the efficiency of delivery of drugs such as antisense oligonucleotides across the blood-brain barrier when conjugated to peptide shuttles is compromised, and hence efficacy reduced, as such complexes typically accumulate in endosomes or lysosomes.

There remains a need for the development of improved peptide shuttles to facilitate the targeted delivery of a variety of active agents to the CNS via the blood-brain barrier and to the eye via the blood-ocular barriers.

### SUMMARY OF THE DISCLOSURE

A first aspect of the present disclosure provides a fusion peptide comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain.

In one embodiment, said fragment comprises the amino acid sequence of SEQ ID NO:2 or SEQ ID NO:25 or a or derivative thereof, or a sequence at least about 80% identical thereto.

In one embodiment, the peptide sequence of (i) comprises or consists of the amino acid sequence of SEQ ID NO:1 or SEQ ID NO:24 or a derivative thereof or a sequence at least about 80% identical thereto.

In one embodiment, the peptide sequence of (i) comprises or consists of the amino acid sequence of SEQ ID NO:2 or SEQ ID NO:25 or a derivative thereof or a sequence at least about 80% identical thereto.

In one embodiment, the peptide sequence of (i) is linked to the endosomal escape domain via a linker.

In particular embodiments, the endosomal escape domain comprises a peptide sequence. For example, the peptide sequence may comprise an N-terminal fragment of the influenza virus hemagglutinin subunit HA2, a GALA peptide or a nuclear localisation sequence. In one embodiment the fragment of HA2 comprises the amino acid sequence of SEQ ID NO:3 or SEQ ID NO:26, a derivative thereof, or a sequence at least about 80% identical thereto. In one embodiment, the GALA peptide comprises the amino acid sequence of SEQ ID NO:4, a derivative thereof, or sequence at least about 80% identical thereto.

In one embodiment, the endosomal escape domain is linked to the N-terminal end of the peptide sequence of (i).

In one embodiment, the fusion peptide comprises a lipid moiety linked to the N-terminal end. In an exemplary embodiment, the lipid moiety comprises a myristoyl group.

In one embodiment, the fusion peptide further comprises an active agent conjugated thereto. In an exemplary embodiment, the endosomal escape domain is located at the N-terminal end of the fusion peptide and the active agent is conjugated to the N-terminal end of the endosomal escape domain. In an exemplary embodiment, the active agent is conjugated between the peptide sequence of (i) and (ii). In another exemplary embodiment the amino acid sequence of SEQ ID NO:1 or 24 or fragment or derivative thereof, or sequence at least about 80% identical thereto, is located at the C-terminal end of the endosomal escape domain, and the active agent is conjugated to the C-terminal end of the sequence of SEQ ID NO:1 or 24 or fragment or derivative thereof, or sequence at least about 80% identical thereto.

In an exemplary embodiment, the fusion peptide of the first aspect comprises (i) a peptide sequence comprising the amino acid sequence of SEQ ID NO:1 or 2, a derivative thereof, or a sequence at least about 80% identical thereto and (ii) a peptide sequence comprising the amino acid sequence of SEQ ID NO:3, a derivative thereof, or a sequence at least about 80% identical thereto. In an exemplary embodiment, the fusion peptide of the first aspect comprises (i) a peptide sequence comprising the amino acid sequence of SEQ ID NO:24 or 25, a derivative thereof, or a sequence at least about 80% identical thereto and (ii) a peptide sequence comprising the amino acid sequence of SEQ ID NO:26, a derivative thereof, or a sequence at least about 80% identical thereto.

Another aspect of the present disclosure provides the use of a fusion peptide comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain, for the targeted delivery of an active agent to the central nervous system (CNS) across the blood-brain barrier or to the eye across a blood-ocular barrier.

The blood-ocular barrier may be the blood-aqueous barrier or the blood-retinal barrier.

The active agent may be linked to component (i) or component (ii) of the fusion peptide. The active agent may be linked to the fusion peptide via a linker.

The active agent may be, for example, a therapeutic agent, a diagnostic agent or an imaging agent. In exemplary embodiments, the active agent may comprise, for example, a proteinaceous molecule or a nucleic acid molecule. The nucleic acid molecule may comprise an antisense oligonucleotide.

A further aspect of the present disclosure provides a method for delivering an active agent across the blood-brain barrier to the CNS, comprising linking the active agent to a fusion peptide according to the first aspect and systemically administering the resultant compound to a subject in need thereof, wherein said administration results in delivery of the active agent across the blood-brain barrier to the CNS.

The active agent may be, for example, a therapeutic agent, a diagnostic agent or detection agent. In exemplary embodiments, the active agent may comprise, for example, a proteinaceous molecule or a nucleic acid molecule.

In an embodiment, the active agent is a therapeutic agent for the treatment of a neurological disorder, a neuromuscular disorder, or other disorder affecting the CNS.

In an embodiment, the active agent is a diagnostic agent for the detection and diagnosis of a neurological or neuromuscular disorder, or other disorder affecting the CNS.

In an embodiment, the active agent is a detection agent for detecting, and optionally imaging, biological tissue within the central nervous system and/or abnormal structures of the central nervous system, in particular the brain.

A further aspect of the present disclosure provides a method for delivering an active agent across a blood-ocular barrier to the eye, comprising linking the active agent to a fusion peptide according to the first aspect and systemically administering the resultant compound to a subject in need thereof, wherein said administration results in delivery of the active agent across a blood-ocular barrier to the eye.

The blood-ocular barrier may be the blood-aqueous barrier or the blood-retinal barrier.

The active agent may be, for example, a therapeutic agent, a diagnostic agent or detection agent. In exemplary embodiments, the active agent may comprise, for example, a proteinaceous molecule or a nucleic acid molecule.

In an embodiment, the active agent is a therapeutic agent for the treatment of an ocular disorder.

In an embodiment, the active agent is a diagnostic agent for the detection and diagnosis of an ocular disorder.

In an embodiment, the active agent is a detection agent for detecting, and optionally imaging, intraocular tissue and/or abnormal structures of the eye.

A further aspect of the present disclosure provides a method for increasing the bioavailability in the CNS or eye of an active agent, comprising linking the active agent to a fusion peptide according to the first aspect and systemically administering the resultant compound to a subject in need thereof, wherein said administration results in an increased bioavailability of the active agent in the CNS or eye, compared to the delivery of the active agent in the absence of the fusion peptide.

A further aspect of the present disclosure provides a method for treating or preventing a disorder of the CNS, or at least one symptom thereof, in a subject, comprising systemically administering to the subject an effective amount of therapeutic agent linked to a fusion peptide according to the first aspect, wherein the peptide conjugate facilitates delivery of the therapeutic agent across the blood-brain barrier to the CNS.

In an embodiment, the disorder of the CNS is a neurological or neuromuscular disorder. In an exemplary embodiment described herein the disorder is spinal muscular atrophy.

A further aspect of the present disclosure provides a method for diagnosing a disorder of the CNS in a subject, comprising systemically administering to the subject a diagnostic agent linked to a fusion peptide according to the first aspect, wherein the fusion peptide facilitates delivery of the diagnostic agent across the blood-brain barrier to the CNS.

A further aspect of the present disclosure provides a method for visualising a region or structure of the CNS, comprising systemically administering to the subject imaging agent comprising a CNS tissue-targeting moiety and a detectable label linked to an imaging moiety and a fusion peptide according to the first aspect, wherein the fusion peptide facilitates delivery of the detection agent across the blood-brain barrier to the CNS.

A further aspect of the present disclosure provides a method for treating or preventing an ocular disorder, or at least one symptom thereof, in a subject, comprising systemically administering to the subject an effective amount of therapeutic agent linked to a fusion peptide according to the first aspect, wherein the peptide conjugate facilitates delivery of the therapeutic agent across a blood-ocular barrier to the eye.

A further aspect of the present disclosure provides a method for diagnosing an ocular disorder in a subject, comprising systemically administering to the subject a diagnostic agent linked to a fusion peptide according to the first aspect, wherein the fusion peptide facilitates delivery of the diagnostic agent across a blood-ocular barrier to the eye.

A further aspect of the present disclosure provides a method for visualising a region or structure of the eye, comprising systemically administering to the subject imaging agent comprising an ocular tissue-targeting moiety and a detectable label linked to an imaging moiety and a fusion peptide according to the first aspect, wherein the fusion peptide facilitates delivery of the detection agent across a blood-ocular barrier to the eye.

A further aspect of the present disclosure provides the use of a peptide comprising or consisting of the amino acid sequence of SEQ ID NO:1 or SEQ ID NO:24, a derivative thereof, or a sequence at least about 80% identical thereto, for the delivery of an active agent to the CNS or eye of a subject.

### BRIEF DESCRIPTION OF THE DRAWINGS

Aspects and embodiments of the present disclosure are described herein, by way of non-limiting example only, with reference to the following drawings.

FIGS. 1. A and B, Schematic representations of exemplary fusion peptides of the present disclosure (SEQ ID NO:6) linked to exemplary active agents: an antisense oligonucleotide targeting the SMN2 gene (A); and the fluorescent dye sulfo-cyanine5.5 (B). C, Schematics of: conjugation of the PMO antisense oligonucleotide exemplified herein at the N-terminus of a TPA-HA2-ApoE fusion peptide (top scheme) to create a linear structure; and conjugation of the PMO antisense oligonucleotide exemplified herein to TPA placed between HA2 and ApoE to create a branched trifunctional construct (bottom scheme). K, lysine residue in bottom scheme.

FIG. 2. Schematic representations of exemplary conjugation chemistries for the generation of branched conjugates. K, lysine residue; C, cysteine residue; X, aminohexanoic acid residue.

FIG. 3. (A) SMN2 pre-mRNA exon-7 skipping and production of 80-90% SMN2Δ7 mRNA transcripts. Peptide-PMO binding to Intron-splicing silencer (ISS-N1) and restoration of SMN2 splicing and production of full-length SMN2 mRNA transcripts. The positions of forward and reverse primers highlighted on full-length mRNA. (B)-(C) Effect of administration of peptides linked to PMO for SMN2 exon 7 inclusion, measured as change in the level of full length SMN2/HPRT1 in SMA patient-derived fibroblasts, each at two concentrations, 0.5 μM and 1 μM (or 1 μM and 5 μM in the case of PMO alone). UT, untreated. (B) For PMO constructs, from left to right: PMO; ApoE(141-150)-PMO; ApoE(133-150)-PMO. (C) For PMO constructs, from left to right: PMO; HA2-PMO; GALA-PMO; NLS-PMO; ApoE(141-150)-PMO; HA2-ApoE(141-150)-PMO; GALA-ApoE(141-150)-PMO; NLS-ApoE(141-150)-PMO; H4-ApoE(141-150)-PMO; A4-ApoE(141-150)-PMO. *P<0.05,**P<0.001, ***P<0.0001 cf. Untreated (control) and PMO (1 and 5 μM); #P<0.001 cf. GALA-/NLS-/H4-ApoE(141-150)-PMO (1 μM), \hat{ }<0.001 cf. ApoE(133-150)-PMO (0.5 and 1 μM), ^{O}P<0.001 cf. ApoE(141-150)-PMO (1 μM), ^{&}P<0.05 cf.

FIG. 4. Representative confocal fluorescence microscopy images of SMA patient-derived fibroblasts treated with 1 μM of Cy5-labelled: (A) ApoE(141-150)-PMO; (B) HA2-ApoE(141-150)-PMO; (C) ApoE(133-150)-PMO; and (D) HA2-ApoE(133-150)-PMO, for 1 hr at 37° C.

FIG. 5. Effect of the endosomal escape domain HA2 and lipidation of peptide-PMO conjugates on the viability of fibroblasts determined using MTS assay. UT, untreated control.

FIG. 6. SMN upregulation in SOD1^{G93A} mice. A, administration regime. B, Full length (FL) SMN2 levels in brain, spinal cord (S. cord), kidney and quadriceps (Quad) as determined by RT-qPCR in untreated mice (left hand columns) and mice treated with SEQ ID NO:6-PMO conjugate (right hand columns) after administration at a dose of 8 mg/kg weekly for five weeks. *, p<0.05; **, p<0.001; ***, p<0.0001 cf. saline-treated group.

FIG. 7. Serum stability of TPA-containing fusion peptides described in Table 3. A, linear (TPA-HA2-ApoE(141-150) (filled squares) and RI-TPA-HA2-ApoE(141-150) (open squares)) and branched (Ac-HA2-(TPA)-ApoE(141-150) (filled circles) and RI-Ac-HA2-(TPA)-ApoE(141-150) (open circles)) fusion peptides comprising HA2-ApoE(141-150). B, linear (TPA-HA2-ApoE(133-150) (filled circles) and RI-TPA-HA2-ApoE(133-150) (diamonds)) and branched (Ac-HA2-(TPA)-ApoE(133-150) (inverted filled triangles) and RI-Ac-HA2-(TPA)-ApoE(133-150) (inverted open triangles)) fusion peptides comprising HA2-ApoE(133-150). In B, filled triangles represent TPA-ApoE(133-150) and open triangles represent RI-TPA-ApoE(133-150). RI, retroinverso peptides.

FIG. 8. RT-qPCR analysis of the level of full-length SMN2 in SMA patient-derived fibroblasts (n=1) at varying concentrations (0.25 μM, 0.5 μM and 1 μM) of TPA-containing fusion peptides described in Table 3. UT, untreated controls. A, HA2-ApoE(141-150)-PMO conjugates. From left to right: UT, untreated control; 0.25 μM TPA-HA2-ApoE(141-150); 0.5 μM TPA-HA2-ApoE(141-150); 1 μM TPA-HA2-ApoE(141-150); 0.25 M branched HA2-TPA-ApoE(141-150); 0.5 μM branched HA2-TPA-ApoE(141-150); 1 M branched HA2-TPA-ApoE(141-150); 0.25 μM RI branched HA2-TPA-ApoE(141-150); 0.5 μM RI branched HA2-TPA-ApoE(141-150); 1 μM RI branched HA2-TPA-ApoE(141-150). RI, retroinverso peptides. B, HA2-ApoE(133-150)-PMO conjugates. From left to right: UT, untreated control; 0.25 μM TPA-ApoE(133-150); 0.5 μM TPA-ApoE(133-150); 1 μM TPA-ApoE(133-150); 0.25 μM TPA-HA2-ApoE(133-150); 0.5 μM TPA-HA2-ApoE(133-150); 1 μM TPA-HA2-ApoE(133-150); 0.25 μM branched HA2-TPA-ApoE(133-150); 0.5 μM branched HA2-TPA-ApoE(133-150); 1 μM branched HA2-TPA-ApoE(133-150); 0.25 μM RI TPA-ApoE(133-150); 0.5 μM RI TPA-ApoE(133-150); 1 μM RI TPA-ApoE(133-150); 0.25 μM RI TPA-HA2-ApoE(133-150); 0.5 μM RI TPA-HA2-ApoE(133-150); RI 1 μM TPA-HA2-ApoE(133-150); 0.25 μM RI branched HA2-TPA-ApoE(133-150); 0.5 μM RI branched HA2-TPA-ApoE(133-150); 1 μM RI branched HA2-TPA-ApoE(133-150). RI, retroinverso peptides.

Amino acid and nucleotide sequences are referred to by a sequence identifier number (SEQ ID NO). Sequences are provided in the Sequence Listing. The amino acid sequence set forth in SEQ ID NO:1 represents a contiguous 18 amino acid sequence from the receptor binding domain of human ApoE (corresponding to amino acids 133 to 150 of the mature ApoE sequence; residues 151-168 of the precursor ApoE sequence in UniProt Accession No. P06249). The sequence of SEQ ID NO:1 is hereinafter referred to as ‘ApoE(133-150)’. The amino acid sequence set forth in SEQ ID NO:2 represents a contiguous 10 amino acid sequence from the receptor binding domain of human ApoE (corresponding to amino acids 141 to 150 of the mature ApoE sequence; residues 159-168 of the precursor ApoE sequence in UniProt Accession No. P06249). The sequence of SEQ ID NO:1 is hereinafter referred to as ‘ApoE(133-150)’. The amino acid sequence set forth in SEQ ID NO:3 represents a 15 amino acid sequence from the N-terminal of the influenza virus hemagglutinin HA2 subunit. The sequence of SEQ ID NO:3 is hereinafter referred to as ‘HA2’. The amino acid sequence set forth in SEQ ID NO:4 represents the synthetic, pH-responsive amphipathic GALA peptide. The amino acid sequence set forth in SEQ ID NO:5 represents an exemplary nuclear localization sequence. The amino acid sequence set forth in SEQ ID NO:6 represents a fusion peptide comprising the sequence of SEQ ID NO:3 conjugated to the N-terminal of SEQ ID NO:1 via a linker. The amino acid sequence set forth in SEQ ID NO:7 represents a fusion peptide comprising the sequence of SEQ ID NO:3 conjugated to the N-terminal of SEQ ID NO:2 via a linker. The nucleotide set forth in SEQ ID NO:8 represents the nusinersen antisense oligonucleotide. The nucleotide sequence set forth in SEQ ID NO:9 represents a 20-mer phosphorodiamidate morpholino oligomer (PMO) exemplified herein. The sequence of SEQ ID NO:9 is hereinafter referred to as ‘PMO’. The sequence of primers used in studies described herein are set forth in SEQ ID NOs:10 to 15. SEQ ID NOs:16-23 represent exemplary fusion peptides of the present disclosure. SEQ ID NOs:24-26 represent D-amino acid-containing retroinverso peptides corresponding to the sequences of SEQ ID NOs:1-3, respectively.

Amino acid and nucleotide sequences described herein, and their respective SEQ ID NOs as per the Sequence Listing, are set out in Table 1 below. Nucleotide sequences are shown 5′-3′.

TABLE 1

| | Sequence | SEQ ID NO |
|---|---|---|
| ApoE(133-150) | LRVRLASHLRKLRKRLLR | 1 |
| ApoE(141-150) | LRKLRKRLLR | 2 |
| HA2 | GLFHAIAHFIHGGWH | 3 |
| GALA | WEAALAEALAEALAEHLAEALAEALEALAA | 4 |
| NLS | VQRKRQKLMP | 5 |
| HA2-ApoE(133-150) | GLFHAIAHFIHGGWHXLRVRLASHLRKLRKRLLR | 6 |
| HA2-ApoE(141-150) | GLFHAIAHFIHGGWHXLRKLRKRLLR | 7 |
| Nusinersen | TCACTTTCATAATGCTGG | 8 |
| PMO | ATTCACTTTCATAATGCTGG | 9 |
| Human SMN2 | GCTTTGGGAAGTATGTTAATTTCA | 10 |
| forward primer | | |
| Human SMN2 | CTATGCCAGCATTTCTCCTTAATT | 11 |
| reverse primer | | |
| Human HPRT1 | GACCAGTCAACAGGGGACAT | 12 |
| forward primer | | |
| Human HPRT1 | CCTGACCAAGGAAAGCAAAG | 13 |
| reverse primer | | |
| Mouse HPRT1 | GATCAGTCAACGGGGGACAT | 14 |
| forward primer | | |
| Mouse HPRT1 | CATTTTGGGGCTGTACTGCTT | 15 |
| reverse primer | | |
| TPA-HA2- | TPA-mPEG-GLFHAIAHFIHGGWH-mPEG-LRVRLASHLRKLRKRLLR | 16 |
| ApoE(133-150) | | |
| Branched HA2- | GLFHAIAHFIHGGWH-mPEG-K(mPEG-TPA)-mPEG-LRVRLASHLRKLRKRLLR | 17 |
| (TPA)-ApoE(133-150) | | |
| TPA-HA2- | TPA-mPEG-GLFHAIAHFIHGGWH-mPEG-LRKLRKRLLR | 18 |
| ApoE(141-150) | | |
| Branched HA2- | GLFHAIAHFIHGGWH-mPEG-K(mPEG-TPA)-mPEG-LRKLRKRLLR | 19 |
| (TPA)-ApoE(141-150) | | |
| RI TPA-HA2- | TPA-mPEG-hwgghifhaiahflg-mPEG-K-mPEG-rllrkrlkrlhsalrvrl | 20 |
| ApoE(133-150) | | |
| RI Branched HA2- | hwgghifhaiahflg-mPEG-K(mPEG-TPA)-mPEG-rllrkrlkrlhsalrvrl | 21 |
| (TPA)-ApoE(133-150) | | |
| RI TPA-HA2- | TPA-mPEG-hwgghifhaiahflg-mPEG-K-mPEG-rllrkrlkrl | 22 |
| ApoE(141-150) | | |
| RI Branched HA2- | hwgghifhaiahflg-mPEG-K(mPEG-TPA)-mPEG-rllrkrlkrl | 23 |
| (TPA)-ApoE(141-150) | | |
| Retroinverso | rllrkrlkrlhsalrvrl | 24 |
| ApoE(133-150) | | |
| Retroinverso | rllrkrlkrl | 25 |
| ApoE(141-150) | | |
| Retroinverso HA2 | hwgghifhaiahflg | 26 |

X = aminohexanoic acid; TPA = thiopropionic acid; mPEG = mini-PEG spacer (amino-PEG2-acetic
acid); K = lysine
RI = retroinverso peptides; D-amino acids shown in lower case
Branched refers to the branched nature of a construct when an active agent is conjugated to
the fusion peptide

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

The term “peptide” means a polymer made up of amino acids linked together by peptide bonds. The term “polypeptide” may also be used to refer to such a polymer although in some instances a polypeptide may be longer (i.e. composed of more amino acid residues) than a peptide. Typically, the term peptide is used to define a sequence of amino acids of up to about 70 amino acids.

The term “fusion peptide” as used herein relates to a peptide comprising two or more heterologous regions or domains not found operably linked in nature.

An “antisense oligonucleotide” refers to a single-stranded oligonucleotide having a sequence that permits hybridization to a corresponding region or segment of a target nucleic acid, e.g. a gene or mRNA. Reference to an antisense oligonucleotide includes reference to both unmodified and modified antisense oligonucleotides, wherein a modified antisense oligonucleotide contains at least one modified nucleoside and/or modified internucleoside linkage.

As used herein the terms “treating”, “treatment”, “preventing”, “prevention” and grammatical equivalents refer to any and all uses which remedy, prevent, retard or delay the establishment of an ocular disorder, a neurological disorder, neuromuscular disorder, or other disorder affecting the CNS, or otherwise prevent, hinder, retard, or reverse the progression of such a disorder. Thus the terms “treating” and “preventing” and the like are to be considered in their broadest context. For example, treatment does not necessarily imply that a patient is treated until total recovery. Where the disorder displays or a characterized by multiple symptoms, the treatment or prevention need not necessarily remedy, prevent, hinder, retard, or reverse all of said symptoms, but may prevent, hinder, retard, or reverse one or more of said symptoms.

As used herein the term “effective amount” includes within its meaning a non-toxic but sufficient amount or dose of an agent or compound to provide the desired effect. The exact amount or dose required will vary from subject to subject depending on factors such as the species being treated, the age, size, weight and general condition of the subject, the severity of the disease or condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

Despite antisense oligonucleotides holding great promise as therapeutics, some practical obstacles remain unresolved, such as inefficient cellular uptake, insufficient release of the antisense oligonucleotide complex from endocytic vesicles and difficulty in crossing biological barriers that limit the efficacy of antisense oligonucleotides particularly in treating CNS diseases. As such, antisense oligonucleotides are mostly delivered directly to the target site by direct injections which in many cases cause severe side-effects. As shown herein, the inventors have identified peptides and fusion peptides capable of crossing the blood-brain barrier. As exemplified herein, fusion peptides of the present disclosure, when conjugated to an antisense oligonucleotide designed to correct the splicing of survival motor neuron-2 (SMN2) mRNA, significantly improved delivery of the antisense oligonucleotide to the brain and significantly increased functional SMN2 levels.

Accordingly, in embodiments of the invention described herein are brain-penetrating peptides and peptide complexes that enhance the efficiency of delivery, and efficacy, of peripherally administered antisense oligonucleotides to CNS targets.

In one aspect the present disclosure provides a fusion peptide comprising (i) a peptide sequence comprising the amino acid sequence of SEQ ID NO:1 or a fragment or derivative thereof, or a sequence at least about 80% identical thereto and (ii) an endosomal escape domain. Optionally, an active agent is conjugated to the fusion peptide as described hereinbelow.

The endosomal escape domain may be linked to the N-terminal or C-terminal end of the peptide sequence of (i) and may be linked directly or indirectly thereto. In particular embodiments the endosomal escape domain is linked to the N-terminal end of the peptide sequence of (i). Typically, the C-terminal end of the endosomal escape domain is linked to the N-terminal end of the peptide sequence of (i).

Components (i) and (ii) may be directly attached to one another or may be attached via one or more linkers or spacers. As used herein, the terms “linker” and “spacer” refer to any molecule or group of molecules that binds or joins two components and may be used interchangeably. Linkers may comprise functional groups enabling the conjugation of active agents to the peptide. Linkers or spacers may provide for optimal spacing of components of the fusion peptide, for example providing flexibility to the construct and enabling each component of the construct, including any active agent conjugated to the fusion peptide, to interact with its respective target, and optimise or increase cell uptake and activity.

Suitable linkers include, by way of example only, amino acids such as aminohexanoic acid, 4-aminobutryic acid, 8-aminooctanoic acid, lysine, glycine and serine and stretches of two or more amino acids such as glycine and serine. Linkers such as aminohexanoic acid and lysine allow for the conjugation of moieties such as active agents to the peptide via an amide bond.

In an exemplary embodiments, fusion peptides of the present disclosure may comprise an aminohexanoic acid moiety between components (i) and (ii), and/or at the C-terminus of the fusion peptide, typically at the C-terminus of the peptide sequence comprising the amino acid sequence of SEQ ID NO:1 or a fragment or derivative thereof, or a sequence at least about 80% identical thereto.

Other suitable linkers may comprise a functional group, optionally a thiol group, to facilitate conjugation of active agents to the peptide. Exemplary thiol group-containing linkers for functionalization of the fusion peptide include, for example, cysteine and thiopropionic acid (TPA). For example a cysteine residue may be incorporated at the N-terminus or C-terminus of a peptide, optionally the C-terminus, to enable “click” conjugation of a moiety such as an oligonucleotide optionally functionalized with maleimide (e.g. Patil et al., 2019, Bioconjug Chem 30:793-799).

In exemplary embodiments, fusion peptides of the present disclosure may comprise a cysteine residue at the C-terminal end of the fusion peptide, typically at the C-terminus of the peptide sequence comprising the amino acid sequence of SEQ ID NO:1 or a fragment or derivative thereof, or a sequence at least about 80% identical thereto. Also in exemplary embodiments, the cysteine residue may be immediately preceded by an aminohexanoic acid moiety.

In exemplary embodiments, fusion peptides of the present disclosure may comprise TPA between components (i) and (ii), and/or at the N-terminus and/or C-terminus of the fusion peptide. In particular exemplary embodiments, TPA is incorporated between components (i) and (ii) of the fusion peptide. In further exemplary embodiments in which the endosomal escape domain is linked to the N-terminal end of the peptide sequence of (i), TPA is incorporated at the N-terminus of the endosomal escape domain.

Linkers may further supply a labile linkage that allows the two components to be separated from each other. Labile linkages include photocleavable groups, acid-labile moieties, base-labile moieties and enzyme-cleavable groups.

Linkers may be incorporated between components of the fusion peptide and/or at the N-terminal and/or C-terminal ends of the fusion peptide to provide flexibility enabling each component the fusion peptide, and optionally an active agent or other moiety conjugated to the fusion peptide access to interact with their respective targets and receptors facilitating cellular uptake and activity. As an alternative, or in addition to one or more of the above-described linkers, moieties of variable length may be employed, including for example polyethylene glycol (PEG) moieties. The PEG moiety may be a short, or ‘mini-PEG’ spacer of 2, 3, 4, 5 or 6 (or longer) unit length. Exemplary mini-PEG spacers are:

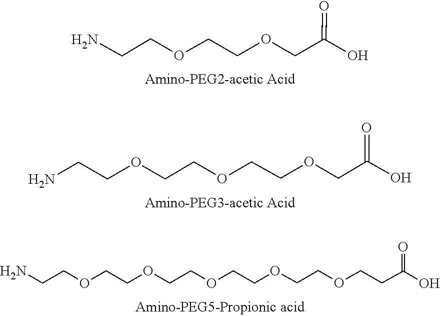 [SMILES: NCCOCCOCC(=O)O ; NCCOCCOCCOCC(=O)O ; NCCOCCOCCOCCOCCOCCC(=O)O]

In exemplary embodiments, fusion peptides of the present disclosure may comprise a mini-PEG moiety, optionally amino-PEG2-acetic acid, between components (i) and (ii), and/or at the N-terminus and/or C-terminus of the fusion peptide. In particular exemplary embodiments, a mini-PEG moiety is incorporated between components (i) and (ii) of the fusion peptide. In further exemplary embodiments in which the endosomal escape domain is linked to the N-terminal end of the peptide sequence of (i), TPA is incorporated at the N-terminus of the endosomal escape domain. In exemplary embodiments in which the fusion peptide comprises TPA between components (i) and (ii), one or more mini-PEG moieties may flank the TPA. In exemplary embodiments in which TPA is incorporated at the N-terminal end of the fusion peptide (typically at the N-terminus of the endosomal escape domain), the TPA may be spaced from the N-terminal amino acid residue by a mini-PEG moiety.

The fusion peptides of the present disclosure comprise a peptide sequence comprising the amino acid sequence of SEQ ID NO:1 or a fragment or derivative thereof, or a sequence at least about 80% identical thereto. A “fragment” of a peptide is a subsequence of the peptide that performs a similar function and retains substantially the same activity as the peptide sequence from which the fragment is derived. In the context of the fusion peptides of the present disclosure, a suitable exemplary fragment comprises the amino acid sequence of SEQ ID NO:2.

The endosomal escape domain comprises a moiety that prevents endosomal entrapment and/or facilitates removal or escape of the fusion peptide, and any agent linked to the fusion peptide, from endosomal compartments or vesicles within endothelial cells following crossing of the blood-brain barrier or a blood-ocular barrier. Typically the endosomal escape domain comprises a pH-dependent endosomal membrane disrupting moiety. In particular embodiments, the endosomal escape domain comprises a peptide sequence. By way of example, the peptide sequence may comprise an N-terminal fragment of the influenza virus hemagglutinin subunit HA2, a GALA peptide or a nuclear localisation sequence. In one exemplary embodiment the fragment of HA2 comprises the amino acid sequence of SEQ ID NO:3, a derivative thereof, or a sequence at least about 80% identical thereto. In one embodiment, the GALA peptide comprises the amino acid sequence of SEQ ID NO:4, a derivative thereof, or sequence at least about 80% identical thereto. In an exemplary embodiment, the nuclear localisation sequence comprises the amino acid sequence of SEQ ID NO:5, a derivative thereof, or sequence at least about 80% identical thereto, although those skilled in the art will appreciate that many other suitable nuclear localisation sequences may be employed. Other suitable endosomal escape domains will also be known to those skilled in the art.

Also contemplated herein are retroinverso analogs of the peptides described above, wherein peptide sequences are synthesised in the reverse order using D-amino acids, ensuring the side chains of the amino acids in the sequence have the same spatial orientation as in peptides generating with natural L-amino acids. Thus, in an embodiment, a retroinverso peptide sequence may be generated based on the peptide sequence comprising the amino acid sequence of SEQ ID NO:1 or a fragment or derivative thereof, or a sequence at least about 80% identical thereto. The peptide sequence set forth in SEQ ID NO:24 represents the retroinverso sequence of the peptide sequence of SEQ ID NO:1. The peptide sequence set forth in SEQ ID NO:25 represents the retroinverso sequence of the peptide sequence of SEQ ID NO:2. In an embodiment, a retroinverso peptide sequence may be generated based on the peptide sequence comprising the endosomal escape domain. The peptide sequence set forth in SEQ ID NO:26 represents the retroinverso sequence of the peptide sequence of SEQ ID NO:3.

Accordingly, an aspect of the present disclosure provides a fusion peptide comprising (i) a peptide sequence comprising the amino acid sequence of SEQ ID NO:24 or 25 or a fragment or derivative thereof, or a sequence at least about 80% identical thereto and (ii) an endosomal escape domain or a retroinverso peptide sequence thereof. The endosomal escape domain or retroinverso sequence thereof may be linked to the N-terminal or C-terminal end of the peptide sequence of (i) and may be linked directly or indirectly thereto. In particular embodiments, the endosomal escape domain or retroinverso sequence thereof is linked to the N-terminal end of the peptide sequence of (i). Typically the C-terminal end of the endosomal escape domain or retroinverso sequence thereof is linked to the N-terminal end of the sequence of (i). In an exemplary embodiment, a fusion peptide may comprise the amino acid sequence of SEQ ID NO:26 or a fragment or derivative thereof, or a sequence at least about 80% identical thereto and the amino acid sequence of SEQ ID NO:24 or a fragment or derivative thereof, or a sequence at least about 80% identical thereto. In another exemplary embodiment, a fusion peptide may comprise the amino acid sequence of SEQ ID NO:26 or a fragment or derivative thereof, or a sequence at least about 80% identical thereto and the amino acid sequence of SEQ ID NO:25 or a fragment or derivative thereof, or a sequence at least about 80% identical thereto.

An exemplary fusion peptide of the present disclosure comprises the amino acid sequence of SEQ ID NO:3 or a fragment or derivative thereof, or a sequence at least about 80% identical thereto, conjugated to the N-terminal end of the amino acid sequence of SEQ ID NO:1 or a fragment or derivative thereof, or a sequence at least about 80% identical thereto, wherein the fusion peptide is capable of being conjugated with an active agent to deliver the active agent across the blood-brain barrier or a blood-ocular barrier. In an embodiment the fusion peptide is acetylated at the N-terminus. In an embodiment the fusion peptide is myristoylated at the N-terminus. In an embodiment the fusion peptide comprises one or more linkers between the amino acid sequence of SEQ ID NO:3 or a fragment or derivative thereof, or a sequence at least about 80% identical thereto and the amino acid sequence of SEQ ID NO:1 or a fragment or derivative thereof, or a sequence at least about 80% identical thereto. In an embodiment said linker comprises an aminohexanoic acid residue. In an embodiment said linker comprises TPA. In an embodiment said linker comprises one or more PEG moieties. In an embodiment said linker comprises TPA and one or more PEG moieties. In an embodiment said linker comprises one or more PEG moieties and a lysine residue, optionally also comprising TPA. In an embodiment the fusion peptide comprises an aminohexanoic acid residue and a cysteine residue at the C-terminal end of the amino acid sequence of SEQ ID NO:1 or a fragment or derivative thereof, or a sequence at least about 80% identical thereto. In an embodiment, the fusion peptide comprises the sequence of SEQ ID NO:6, 16 or 17.

An exemplary fusion peptide of the present disclosure comprises the amino acid sequence of SEQ ID NO:3 or a fragment or derivative thereof, or a sequence at least about 80% identical thereto, conjugated to the N-terminal end of the amino acid sequence of SEQ ID NO:2 or a fragment or derivative thereof, or a sequence at least about 80% identical thereto, wherein the fusion peptide is capable of being conjugated with an active agent to deliver the active agent across the blood-brain barrier or a blood-ocular barrier. In an embodiment the fusion peptide is acetylated at the N-terminus. In an embodiment the fusion peptide is myristoylated at the N-terminus. In an embodiment the fusion peptide comprises one or more linkers between the amino acid sequence of SEQ ID NO:3 or a fragment or derivative thereof, or a sequence at least about 80% identical thereto and the amino acid sequence of SEQ ID NO:2 or a fragment or derivative thereof, or a sequence at least about 80% identical thereto. In an embodiment said linker comprises an aminohexanoic acid residue. In an embodiment said linker comprises TPA. In an embodiment said linker comprises one or more PEG moieties. In an embodiment said linker comprises TPA and one or more PEG moieties. In an embodiment said linker comprises one or more PEG moieties and a lysine residue, optionally also comprising TPA. In an embodiment the fusion peptide comprises an aminohexanoic acid residue and a cysteine residue at the C-terminal end of the amino acid sequence of SEQ ID NO:2 or a fragment or derivative thereof, or a sequence at least about 80% identical thereto. In an embodiment, the fusion peptide comprises the sequence of SEQ ID NO:7, 18 or 19.

An exemplary fusion peptide of the present disclosure comprises the amino acid sequence of SEQ ID NO:26 or a fragment or derivative thereof, or a sequence at least about 80% identical thereto, conjugated to the N-terminal end of the amino acid sequence of SEQ ID NO:24 or a fragment or derivative thereof, or a sequence at least about 80% identical thereto, wherein the fusion peptide is capable of being conjugated with an active agent to deliver the active agent across the blood-brain barrier or a blood-ocular barrier. In an embodiment the fusion peptide is acetylated at the N-terminus. In an embodiment the fusion peptide is myristoylated at the N-terminus. In an embodiment the fusion peptide comprises one or more linkers between the amino acid sequence of SEQ ID NO:26 or a fragment or derivative thereof, or a sequence at least about 80% identical thereto and the amino acid sequence of SEQ ID NO:24 or a fragment or derivative thereof, or a sequence at least about 80% identical thereto. In an embodiment said linker comprises an aminohexanoic acid residue. In an embodiment said linker comprises TPA. In an embodiment said linker comprises one or more PEG moieties. In an embodiment said linker comprises TPA and one or more PEG moieties. In an embodiment said linker comprises one or more PEG moieties and a lysine residue, optionally also comprising TPA. In an embodiment the fusion peptide comprises an aminohexanoic acid residue and a cysteine residue at the C-terminal end of the amino acid sequence of SEQ ID NO:24 or a fragment or derivative thereof, or a sequence at least about 80% identical thereto. In an embodiment, the fusion peptide comprises the sequence of SEQ ID NO:20 or 21.

An exemplary fusion peptide of the present disclosure comprises the amino acid sequence of SEQ ID NO:26 or a fragment or derivative thereof, or a sequence at least about 80% identical thereto, conjugated to the N-terminal end of the amino acid sequence of SEQ ID NO:25 or a fragment or derivative thereof, or a sequence at least about 80% identical thereto, wherein the fusion peptide is capable of being conjugated with an active agent to deliver the active agent across the blood-brain barrier or a blood-ocular barrier. In an embodiment the fusion peptide is acetylated at the N-terminus. In an embodiment the fusion peptide is myristoylated at the N-terminus. In an embodiment the fusion peptide comprises one or more linkers between the amino acid sequence of SEQ ID NO:26 or a fragment or derivative thereof, or a sequence at least about 80% identical thereto and the amino acid sequence of SEQ ID NO:25 or a fragment or derivative thereof, or a sequence at least about 80% identical thereto. In an embodiment said linker comprises an aminohexanoic acid residue. In an embodiment said linker comprises TPA. In an embodiment said linker comprises one or more PEG moieties. In an embodiment said linker comprises TPA and one or more PEG moieties. In an embodiment said linker comprises one or more PEG moieties and a lysine residue, optionally also comprising TPA. In an embodiment the fusion peptide comprises an aminohexanoic acid residue and a cysteine residue at the C-terminal end of the amino acid sequence of SEQ ID NO:25 or a fragment or derivative thereof, or a sequence at least about 80% identical thereto. In an embodiment, the fusion peptide comprises the sequence of SEQ ID NO:22 or 23.

Embodiments of the disclosure contemplate derivatives of peptide sequences disclosed herein. As used herein the term “derivative” is intended to encompass chemical modification to a peptide or one or more amino acid residues of a peptide, including chemical modification in vitro, for example by introducing a group in a side chain in one or more positions of a peptide, such as a nitro group in a tyrosine residue or iodine in a tyrosine residue, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain. Other derivatives may be obtained by oxidation or reduction of the side-chains of the amino acid residues in the peptide. Modification of an amino acid may also include derivation of an amino acid by the addition and/or removal of chemical groups to/from the amino acid, and may include substitution of an amino acid with an amino acid analog (such as a phosphorylated or glycosylated amino acid) or a non-naturally occurring amino acid such as a N-alkylated amino acid (e.g. N-methyl amino acid), D-amino acid, β-amino acid or γ-amino acid.

Also contemplated herein are conservative variants of the peptide sequences disclosed herein. Conservative variants comprise one or more conservative amino acid substitutions. A “conservative amino acid substitution” is one in which an amino acid residue is replaced with another residue having a chemically similar or derivatised side chain. Families of amino acid residues having similar side chains, for example, have been defined in the art (e.g. see Lehninger A. L., Biochemistry, second ed., pp. 73-75, Worth Publishers, New York (1975). These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Conservative substitutions will entail exchanging a member of one of these classes for a member of the same class. For example, the substitution of the neutral amino acid serine (S) for the similarly neutral amino acid threonine (T) would be a conservative amino acid substitution. Those skilled in the art will be able to determine suitable conservative amino acid substitutions that do not eliminate the functional properties of the peptide sequence required in the context of the present disclosure.

Variants of the peptide sequences defined herein are contemplated. In particular embodiments, the variant will possess at least about 80% identity to the sequence of which it is a variant. The sequence may be about 80%, 81%, 82%, 83%, 84%, 85%, 86%, 87%, 88%, 89%, 90%, 91%, 92%, 93%, 94%, 95%, 96%, 97%, 98% or 99% identical to the sequence of which it is a variant. Numerous means are available, and will be known, to those skilled in the art for determining sequence identity, for example computer programs that employ algorithms such as BLAST (Basic Local Alignment Search Tool, Altschul et al., 1993, J. Mol. Biol. 215:403-410).

A fusion peptide of the present disclosure may further comprise one or more additional moieties. For example, a lipid moiety, such as a myristoyl group, may be attached to the N-terminal end of the fusion peptide, optionally to the N-terminal end of the endosomal escape domain. The conjugate may comprise a moiety to facilitate or enhance uptake by brain cells, such as a folate moiety (for example 5-methyltetrahydrofolate) to bind to the folate receptor or sugar moiety (for example glucose or N-acetyl-galactosamine) to bind to class I hexose (glucose) transporters.

The fusion peptide may be linked to a cell-penetrating peptide that is effective to enhance transport of the compound, and any active agent linked thereto, into cells. The cell-penetrating peptide can be attached to either terminus of the fusion peptide, resulting in increased penetration into cells. For example, the cell-penetrating peptide may be an arginine-rich peptide transporter, Penetratin or the Tat peptide. These peptides are well known in the art and are disclosed, for example, in US Publication No. 20100016215.

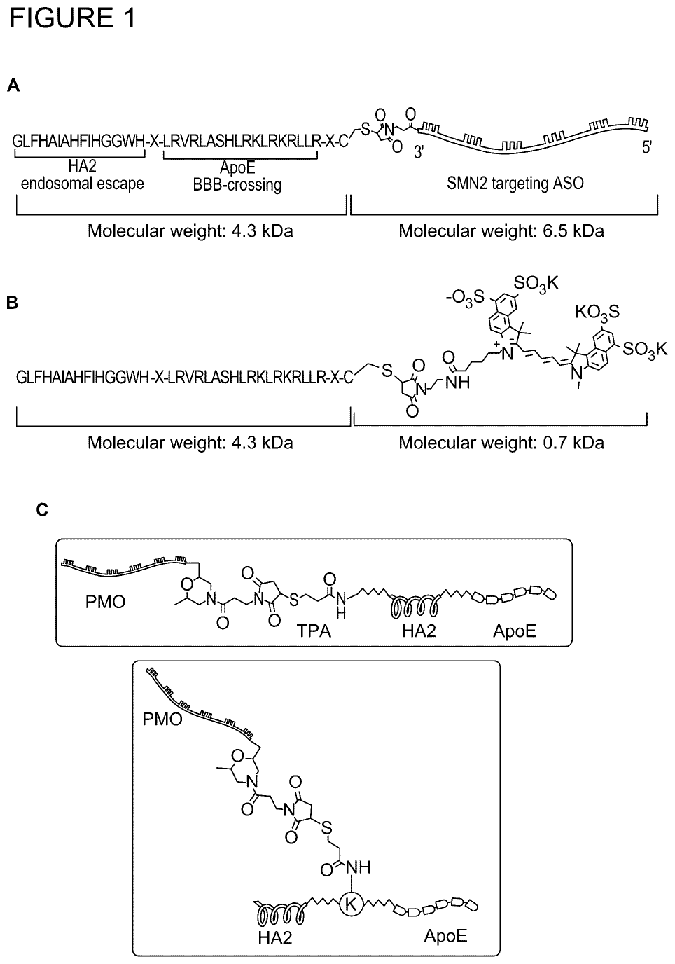

For practical application in accordance with embodiments of the present disclosure, a fusion peptide of the present disclosure may comprise an active agent attached to either terminus. The active agent may be, for example, a therapeutic agent, a diagnostic agent, a detection agent or an agent for imaging biological tissue. Suitable active agents are described hereinbelow and include nucleic acid-based constructs such as antisense oligonucleotides and protein-, polypeptide- or peptide-based moieties. Schematic representations of exemplary conjugate compounds are shown in FIGS. 1A and 1B. The fusion peptide may comprise a suitable lipid moiety, such as a myristoyl group, at the opposite end to which the active agent is attached. In an exemplary embodiment, the active agent is attached to the fusion peptide via a non-reducible bond, and the lipid moiety is attached to the other end of the fusion peptide via a reducible (e.g. disulphide) bond. Without wishing to be bound by theory, the inventors suggest that the resultant compound forms a micelle with the active agent located between the lipid at the core of the micelle and the fusion peptide on the outer surface layer enabling transcytosis across the blood-brain barrier. Once the micelle is taken up by the CNS endothelial cells the bond between the fusion peptide and the lipid moiety will be reduced, resulting in disassembly of the micelle and release of the active agent attached to the fusion peptide.

Provided herein are conjugates comprising fusion peptides of the present disclosure to which one or more active agents are conjugated. The active agent may comprise, for example, a therapeutic agent, a diagnostic agent, a detection agent or an agent for imaging biological tissue. The active agent may be, for example, a nucleic acid-based construct such as an antisense oligonucleotide or a protein-, polypeptide- or peptide-based moiety.

The active agent may be conjugated to the fusion peptide in any suitable orientation. For example, the active agent, such as an antisense oligonucleotide, may be conjugated to one end of the fusion peptide creating a linear conjugate, or alternatively may be conjugated between the endosomal escape domain and the peptide sequence comprising the amino acid sequence of SEQ ID NO:1 or SEQ ID NO:24 or a fragment or derivative thereof, or a sequence at least about 80% identical thereto, thereby generating a branched conjugate. Schematic examples of linear and branched conjugates are shown in FIG. 1C. For linear and branched conjugates, the active agent may be conjugated to the fusion peptide via, for example, a linker such as a cysteine residue or TPA.

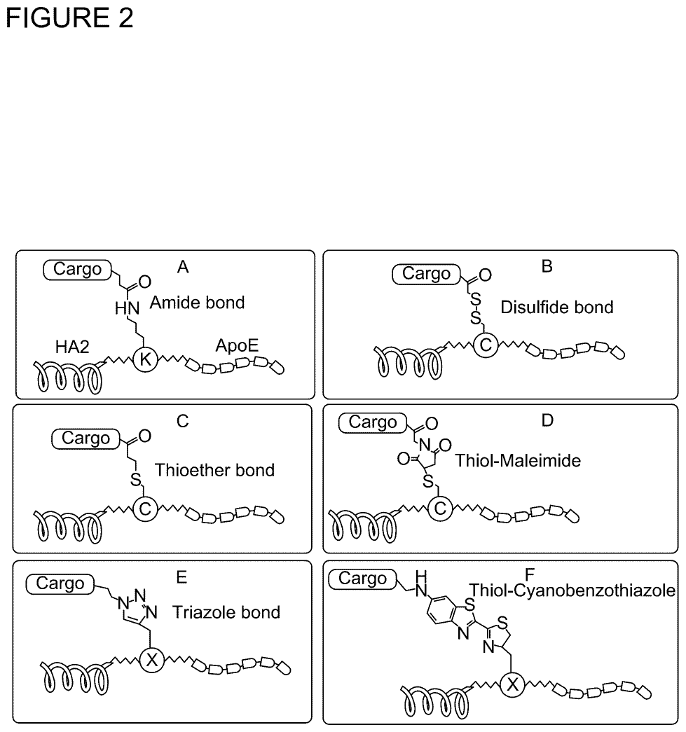

Method of conjugating active agents to the fusion peptides can be achieved using a variety of conjugation chemistry techniques that will be well known to those skilled in the art. By way of example, suitable techniques are described in: Karas et al., 2018, Methods Mol Biol 1828:355-363; Patil et al., 2019, Bioconjug Chem 30:793-799; Shabanpoor and Gait, 2013, Chem Commun (Camb) 49:10260-10262; and Shabanpoor et al., 2015, Nucleic Acids Res 43:29-39. To facilitate the generation of branched conjugates, in which the active agent is conjugated between the endosomal escape domain and the peptide sequence comprising the amino acid sequence of SEQ ID NO:1 or SEQ ID NO:24 or a fragment or derivative thereof, or a sequence at least about 80% identical thereto, the fusion peptide may include an amino acid residue, such as lysine, cysteine or aminohexanoic acid, to facilitate conjugation, optionally via a functional group such as TPA. Examples of suitable conjugation chemistries for the generation of branched conjugates are shown in FIG. 2. The skilled addressee will appreciate that these are merely exemplary, and the scope of the present disclosure is not limited by reference to any specific conjugation chemistry approaches.

Where the active agent is a peptide sequence, synthesis compatibilities mean that the conjugate can be synthesized with the fusion peptide as a single construct by either synthesizing the active agent peptide at the C-terminal or N-terminal end of the fusion peptide. In such cases the conjugate may also be synthesized so as to contain a linker, such as comprising one or more amino acid residues, between the components (i) and (ii) of the fusion peptide (as described above) and/or between the fusion peptide and the active agent peptide.

The peptide components of fusion peptides of the present disclosure may be produced using any method known in the art, including synthetically or by recombinant techniques such as expression of nucleic acid constructs encoding the components. For example a peptide may be synthesized using the Fmoc-polyamide mode of solid-phase peptide synthesis. Other synthesis methods include solid phase t-Boc synthesis and liquid phase synthesis. Purification can be performed by any one, or a combination of, techniques such as re-crystallization, size exclusion chromatography, ion-exchange chromatography, hydrophobic interaction chromatography and reverse-phase high performance liquid chromatography using, for example, acetonitrile/water gradient separation.

A fusion peptide of the present disclosure may be produced when two or more heterologous nucleotide sequences encoding each component of the fusion peptide, optionally including nucleotides sequences encoding a linker or spacer amino acid(s), are fused together in the correct translational reading frame and are expressed. Accordingly, the present disclosure also provides isolated nucleic acid molecules encoding peptides and fusion peptides and components thereof as described herein. Where the active agent to be conjugated to the fusion peptide is a protein-, polypeptide- or peptide-based moiety, a nucleotide sequence(s) encoding the active agent may be operably linked to nucleotide sequences encoding the fusion peptide or a component thereof in a nucleic acid molecule, such that expression of the nucleic acid molecule generates the active agent linked to the appropriate component of the fusion peptide.

The present disclosure also provides vectors comprising a nucleotide sequence(s) encoding peptide sequences and fusion peptides described herein. Typically the nucleotide sequence(s) is operably linked to a promoter to allow for expression of the peptide or fusion peptide. The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art.

Fusion peptides of the present disclosure are particularly suited for use as vehicles or shuttles to deliver active agents across the blood-brain barrier to the CNS or across a blood-ocular barrier to the eye. Accordingly, one aspect of the disclosure provides the use of a fusion peptide comprising (i) a peptide sequence comprising the amino acid sequence of SEQ ID NO:1 or a fragment or derivative thereof, or a sequence at least about 90% identical thereto and (ii) an endosomal escape domain, for the targeted delivery of an active agent to the CNS across the blood-brain barrier or to the eye across a blood-ocular barrier (the blood-aqueous barrier or blood-retinal barrier).

Another aspect of the disclosure provides a method for delivering an active agent across the blood-brain barrier to the CNS or across a blood-ocular barrier to the eye, comprising linking the active agent to a fusion peptide and systemically administering the resultant compound to a subject in need thereof, wherein said administration results in delivery of the active agent across the blood-brain barrier to the CNS or across a blood-ocular barrier to the eye, and wherein the fusion peptide comprises (i) a peptide sequence comprising the amino acid sequence of SEQ ID NO:1 or SEQ ID NO:24 or a fragment or derivative thereof, or a sequence at least about 90% identical thereto and (ii) an endosomal escape domain.

A further aspect provides a method for increasing the bioavailability in the CNS or the eye of an active agent, comprising linking the active agent to a fusion peptide and systemically administering the resultant compound to a subject in need thereof, wherein said administration results in an increased bioavailability of the active agent in the CNS or the eye, compared to the delivery of the active agent in the absence of the fusion peptide, and wherein the fusion peptide comprises (i) a peptide sequence comprising the amino acid sequence of SEQ ID NO:1 or SEQ ID NO:24 or a fragment or derivative thereof, or a sequence at least about 90% identical thereto and (ii) an endosomal escape domain.

Any suitable active agents may be linked or attached to the fusion peptides of the present disclosure. The terms “linked” and “attached” are used interchangeably and relate to any type of interaction that conjugate (join) two entities and include covalent bonds or non-covalent bonds, such as, for example, hydrophobic/hydrophilic interactions, van der Waals forces, ionic bonds, disulphide bonds or hydrogen bonds. In an exemplary embodiment, a cysteine residue is introduced at the C-terminal of the blood-brain or blood-ocular barrier crossing peptide to enable attachment of the active agent via a reducible disulphide bond. Also in an exemplary embodiment a linker may be introduced between the C-terminal residue of the blood-brain or blood-ocular barrier crossing peptide and the aforementioned cysteine residue.

In the context of the present disclosure an active agent is any agent that is biologically active and/or enables or facilitates a biological response and/or beneficial outcome. By way of example, the active agent may be a therapeutic agent, a diagnostic or detection agent or an agent for imaging biological tissue. The active agent may take any suitable form, such as a peptide-, polypeptide- or protein-based molecule, a nucleic acid-based molecule or other organic or inorganic molecule or compound. The fusion peptides of the present disclosure allow for the carriage of active agents of a range of molecular sizes across the blood-brain barrier. The active agent may be, for example up less than about 500 Daltons in size, or may be up to about 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa or 20 kDa.

The active agent may be a therapeutic agent suitable for use in the treatment or prevention of any disease or disorder, wherein the site of action of the agent is within the central nervous system, requiring that the agent crosses the blood-brain barrier, or wherein the site of action of the agent is within the eye, requiring that the agent crosses a blood-ocular barrier. The skilled person will appreciate that the scope of the present disclosure is not limited by reference to any specific type or identity of therapeutic agent. Typically the disease or disorder is a disease or disorder affecting, or that is affected by, the CNS (a CNS disorder) or is an ocular disease or disorder. For example, the CNS disorder may be a neurological or neuromuscular disorder.

Accordingly, an aspect of the present disclosure provides a method for treating or preventing a CNS disorder, or at least one symptom thereof, in a subject, comprising systemically administering to the subject a therapeutic agent linked to a fusion peptide, wherein the fusion peptide comprises (i) a peptide sequence comprising the amino acid sequence of SEQ ID NO:1 or SEQ ID NO:24 or a fragment or derivative thereof, or a sequence at least about 90% identical thereto and (ii) an endosomal escape domain, and wherein the fusion peptide facilitates delivery of the therapeutic agent across the blood-brain barrier to the CNS.

CNS disorders applicable to the present disclosure include, but are not limited to, neurological disorders, neuromuscular disorders and lysosomal storage diseases. Neurological or neuromuscular disorders against which the therapeutic agent may be directed include, by way of example only, spinal muscle atrophy, amyotrophic lateral sclerosis, epilepsy, seizures, stroke, Parkinson's disease, multiple sclerosis, brain tumours such as glioblastoma, dementia including Alzheimer's disease, Huntington's disease, ankylosing spondylitis, spinal stenosis, spina bifida and other spinal disorders, autism, depression, anxiety, bipolar disorder, schizophrenia, disorders associated with or resulting from head trauma, inflammatory disorders, and infections and diseases associated with infection.

Also provided herein is a method for treating or preventing an ocular disorder, or at least one symptom thereof, in a subject, comprising systemically administering to the subject a therapeutic agent linked to a fusion peptide, wherein the fusion peptide comprises (i) a peptide sequence comprising the amino acid sequence of SEQ ID NO:1 or SEQ ID NO:24 or a fragment or derivative thereof, or a sequence at least about 90% identical thereto and (ii) an endosomal escape domain, and wherein the fusion peptide facilitates delivery of the therapeutic agent across a blood-ocular barrier to the eye.

Ocular disorders applicable to the present disclosure include disorders and diseases of the anterior and posterior segments of the eye. Exemplary disorders and diseases include, but are not limited to, retinoptahies such as diabetic retinopathy, macular degeneration such as age-related macular degeneration, glaucoma, dry eye, cytomegalovirus retinitis, keratius-induced corneal neovasculariza Lion, inherited retinal diseases and other ocular vascular and inflammatory diseases.

Exemplary therapeutic agents that may be used in accordance with aspects and embodiments of the present disclosure include antisense nucleic acid molecules. An antisense molecule comprises a nucleotide sequence complementary to a target nucleotide sequence, wherein the antisense molecule modulates the expression or activity of the target sequence. This modulation may comprise inhibiting or increasing, at least partially, the expression of a gene or protein encoded by the target of the antisense molecule or by a region adjacent (upstream or downstream) and operably linked to the target of the antisense molecule. Binding of an antisense molecule to its complementary cellular nucleotide sequence may affect transcription, RNA processing, transport, and/or stability of the miRNA to which it is specific. An antisense molecule may comprise, for example, DNA, RNA, locked nucleic acid (LNA), peptide nucleic acid (PNA) or any combination thereof. Suitable antisense molecules for use in accordance with embodiments disclosed herein include, for example, antisense oligonucleotides, small interfering RNAs (siRNAs) and catalytic antisense nucleic acid constructs.

Exemplary antisense molecules are antisense oligonucleotides. An “antisense oligonucleotide” refers to a single-stranded oligonucleotide having a sequence that permits hybridization to a corresponding region or segment of a target nucleic acid. Reference to an antisense oligonucleotide includes reference to both unmodified and modified antisense oligonucleotides, wherein a modified antisense oligonucleotide contains at least one modified nucleoside and/or modified internucleoside linkage. Exemplary antisense oligonucleotides include phosphorodiamidate morpholino oligomer (PMO or morpholino) oligonucleotides.

Significant advances in recent years have seen the development of numerous promising candidate antisense oligonucleotides for treating various neurological, neuromuscular and ocular disorders. For example, nusinersen (Spinraza®) is an FDA-approved antisense oligonucleotide with the sequence TCACTTTCATAATGCTGG (SEQ ID NO:8) for the treatment of spinal muscular atrophy. Other antisense oligonucleotides are also being developed for treating spinal muscular atrophy, including PMO oligonucleotides (such as that represented in SEQ ID NO:9 for correcting the splicing of the SMN2 gene to include exon 7). Such antisense oligonucleotides may be employed in accordance with the present disclosure.

Other exemplary antisense oligonucleotides suitable for use in accordance with the present disclosure may be designed for the treatment of amyotrophic lateral sclerosis, including those targeting the SOD1 gene (for example as described in Nizzardo et al., 2016, Scientific Reports 6:21301 and McCampbell et al., 2018, J Clin Invest 128:3558-3567) or ataxin-2 (such as those described in Becker et al., 2017, Nature 544:367-371).

Also by way of example, the antisense oligonucleotide may target a gene associated with epilepsy, such as gain of function mutations in the KCNT1 gene associated with particular forms of epilepsy, including epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome. Exemplary antisense oligonucleotides targeting KCNT1 are described in WO 2018/227247, the disclosure of which is incorporated herein by reference. The skilled person will appreciate that the scope of the present disclosure is not limited by reference to any specific antisense oligonucleotides.

The therapeutic agent may be a peptide-, polypeptide- or protein-based agent. The agent may be a small molecule or a large molecule therapeutic. Exemplary therapeutics are agonists of cellular receptors for example neurotensin, cholecystokinin, neuropeptide Y and oxytocin receptors, antagonists of cellular receptors such as NMDA-glutamate receptors, or inhibitors of enzymes such as inhibitors of Cdk5 kinase and γ-secretase or the presenilin subunit thereof. The skilled person will appreciate that the scope of the present disclosure is not limited by reference to any specific small molecule or large molecule agents or any specific peptide-polypeptide- or protein-based agents.

The active agent employed in accordance with embodiments of the present disclosure may be a diagnostic agent, suitable for detecting abnormalities in the CNS or the eye, and hence useful for detecting or diagnosing a neurological, neuromuscular or ocular disorder. By way of example only, the agent may be a peptide, polypeptide or protein that binds to abnormal structures, such as β-amyloid plaques in the brains of Alzheimer's disease sufferers. Alternatively, for example, the agent may be an imaging agent for detecting and visualising CNS or ocular tissue and/or abnormalities in the CNS or eye, wherein the imaging agent comprises a moiety that binds to specific CNS or ocular tissue and a detectable label. Suitable detectable labels include, for example, radio-isotopes, imaging dyes, and paramagnetic material. Suitable imaging techniques will be known to persons skilled in the art, illustrative examples of which include single photon emission computed tomography, positron emission tomography (PET); near infrared fluorescence imaging, ultrasound imaging and magnetic resonance imaging. In an embodiment disclosed herein, the method for detecting the detectable label is selected from the group consisting of: single photon emission computed tomography; positron emission tomography; near infrared fluorescence imaging; ultrasound imaging; and magnetic resonance imaging.

Fusion peptides of the present disclosure with an active agent attached may be formulated into suitable pharmaceutical compositions for in vivo administration, which compositions typically comprise one or more pharmaceutically acceptable carriers, excipients or diluents suitable for systemic administration. Systemic administration can be achieved through any suitable route, including, but not limited to, intravenous, intramuscular, intra-arterial and oral.

It will be understood that the specific dose level of a composition for any particular subject will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable, and may be determined by the skilled addressee without undue burden. Considering a patient, for example, from about 0.1 mg to about 10 mg of agent may be administered per kilogram of body weight per day, per week or per month. Dosage regimens may be adjusted to provide the optimum therapeutic response. For example, several divided doses may be administered daily, weekly, monthly or other suitable time intervals or the dose may be proportionally reduced as indicated by the exigencies of the situation.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

The present disclosure contemplates combination therapies, wherein active agents delivered as described herein are co-administered with other suitable agents that may facilitate the desired therapeutic or prophylactic outcome. By “co-administered” is meant simultaneous administration in the same formulation or in two different formulations via the same or different routes or sequential administration by the same or different routes. By “sequential” administration is meant a time difference of from seconds, minutes, hours or days between the administration of the agents. Administration may be in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### Example 1—Peptide and Peptide-PMO Conjugate Syntheses

Peptide synthesis. Peptides were assembled on solid support using 9-Fluroenylmethoxycarbonyl (Fmoc) protected L-α-amino acids. Peptides were functionalized at the C-terminus with a cysteine residue to enable “click” conjugation to the 3′-end of a PMO (see below) functionalized with maleimide. A spacer (X: aminohexanoic acid) was placed between the C-terminal Cysteine and also within the sequence to separate the blood-brain barrier crossing peptide from the endosomal escape domain. Some peptides were acetylated at the N-terminus with an alkyne to enable fluorescent-labelling of the peptide-PMO conjugate with Cy5-azide.

All peptides were assembled on a CEM Liberty™ Blue microwave peptide synthesizer on a 0.1 mmol scale using TentaGel® XV resin (100-200 mesh, loading: 0.20 mmol/g). The coupling of Fmoc-amino acids (4-fold excess) were carried using 4-fold excess of N,N′-Diisopropylcarbodiimide (DIC) and Ethyl cyano(hydroxyimino)acetate (Oxyma Pure) at 90° C. for 5 min in DMF. Fmoc-Arg(pbf)-OH was double coupled and the Fmoc-Cys(Trt)-OH and Fmoc-His(Trt)-OH were coupled at 50° C. for 10 min. The N-terminal Fmoc-protecting groups were removed by treating the resin-attached peptide with piperidine (20% v/v) in Dimethylformamide (DMF). The deprotection was carried out at 75° C. using 25 W microwave power for 5 min. The N-terminus of the solid phase-bound peptide was acetylated with acetic anhydride (10 equiv.) and DIEA (10 equiv.). The resin-bound polypeptide chain was cleaved from solid support by treatment with a cocktail of Trifluoroacetic acid (TFA): 3,6-dioxa-1,8-octanedithiol (DODT): H2O: Triisopropylsilane (TIPS) (94%:2.5%:2.5%:1%), 20 ml) for 2 hr at room temperature. Excess TFA was evaporated off by blowing nitrogen into the peptide-TFA solution. The cleaved peptide was precipitated in ice cold diethyl ether and centrifuged at 3000 rpm for 3 min. The pellet was washed by re-suspending it in ice-cold diethyl ether and centrifuged again. This washing process was repeated at least three times. Crude peptides were dissolved in water, analysed and purified by RP-HPLC on Phenomenex Jupiter columns (4.6×250 mm, C18, 5 μm) and (21.2×150 mm, C18, 5 μm), respectively, using two different buffer systems, 0.1% TFA in water (solvent A) and 0.1% TFA in 100% acetonitrile (solvent B). A gradient of 10-50 and 10-70% B over 30 min was used at flow rate of 1.5 ml min⁻¹ for the analytical and 10 ml min⁻¹ for the preparative column. All peptides were purified to greater than 90% purity as determined by analytical RP-HPLC.

Functionalisation of PMO. The 20-mer intron-splicing silencer (ISS-N1)-targeting PMO 5′-ATTCACTTTCATAATGCTGG-3′ (SEQ ID NO:9) was purchased from Gene Tools LLC (Philomath, USA). The unmodified PMO was functionalised by coupling 3-maleimido-propionic acid to the free secondary amine group at the 3′-end as previously described (Patil et al., 2019, Bioconjug Chem 20:793-799). Briefly, the 3-maleimido-propionic acid (2-fold excess over PMO) was activated using a 2-fold excess of HBTU and HOAt in NMP in the presence of 5 eq. of DIEA and added to the PMO dissolved in dimethylsulfoxide (DMSO). The reaction was carried out at 40° C. for 2 hr. Purification of the maleimido-PMO conjugate was carried out using RP-HPLC as described above.

The term “PMO” used in these Examples refers to the PMO molecule with the sequence of SEQ ID NO:9 and functionalised as described above.

Peptide-PMO Conjugation. Peptides with cysteine at their C-terminus were conjugated to the 3′-maleimide-functionalised PMO using a thiol-maleimide click reaction. The peptides (200 nmol) and PMO (100 nmol) were each dissolved in PBS (pH 7.4) and mixed. The pH of the reaction was adjusted to 7-8 using NH₄HCO₃. The reaction was monitored by MALDI-TOF mass spectrometry and the absence of PMO indicated the completion of reaction. The peptide-PMO conjugates were purified as described above. Myristic acid was conjugated at the N-terminus of some peptide-PMO conjugates.

Fluorescent labelling. Cy5 azide was coupled in solution to the alkyne at the N-terminus of peptide-PMO conjugates as previously described (Shabanpoor et al., 2013, Chem Commun 49:10260-10262). Briefly, the peptide-PMO conjugates (50 nmol) were dissolved in dH₂O, and Cy5 azide (150 nmol dissolved in DMSO) was added in presence of CuSO₄ (10 eq) and sodium ascorbate (12 eq). The reaction mixture was incubated at 50° C. for 2 hr. The peptide-PMO conjugates were purified as described above. The molar absorption at 265 nm in 0.1 M HCl solution was measured and used to calculate the molar concentration of peptide-PMO conjugates for their subsequent use in cellular and in vivo assays.

According to the procedures described above, the inventors developed a series of peptide conjugates attached to the PMO oligonucleotide of SEQ ID NO:9. The constructs are listed in Table 2:

TABLE 2

| | | [M + H] | | Yield |
|---|---|---|---|---|
| Construct | Sequence | Calc. | Exp. | (%) |
| HA2-PMO | Ac-GLFHAIAHFIHGGWH-X-C-PMO | 8863.3 | 8863.9 | 73 |
| GALA-PMO | Ac-WEAALAEALAEALAEHLAEALAEALEALAA-X-C-PMO | 10195.7 | 10196.5 | 45 |
| NLS-PMO | Ac-VQRKRQKLMP-X-C-PMO | 8446.8 | 8447.6 | 66 |
| ApoE(141-150)-PMO | Ac-LRKLRKRLLR-X-C-PMO | 8513.9 | 8514.8 | 81 |
| HA2-ApoE(141-150)-PMO | Ac-GLFHAIAHFIHGGWH-X-LRKLRKRLLR-X-C-PMO | 10308.8 | 10309.3 | 58 |
| GALA-ApoE(141-150)-PMO | Ac-WEAALAEALAEALAEHLAEALAEALEALAA-X-LRKLR | 11641.3 | 11642.7 | 37 |
| | KRLLR-X-C-PMO | | | |
| NLS-ApoE(141-150)-PMO | Ac-VQRKRQKLMP-X-LRKLRKRLLR-X-C-PMO | 9892.5 | 9894.1 | 65 |
| H4-ApoE(141-150)-PMO | Ac-HHHH-X-LRKLRKRLLR-X-C-PMO | 9175.5 | 9176.4 | 68 |
| A4-ApoE(141-150)-PMO | Ac-AAAA-X-LRKLRKRLLR-X-C-PMO | 8911.2 | 8912 | 55 |
| Myr-ApoE(141-150)-PMO | Myr-LRKLRKRLLR-X-C-PMO | 8683.4 | 8684 | 61 |
| Myr-HA2-ApoE(141-150)-PMO | Myr-GLFHAIAHFIHGGWH-X-LRKLRKRLLR-X-C-PMO | 10477.1 | 10477.9 | 40 |
| ApoE(133-150)-PMO | Ac-LRVRLASHLRKLRKRLLR-X-C-PMO | 9447.1 | 9448.3 | 69 |
| HA2-ApoE(133-150)-PMO | Ac-GLFHAIAHFIHGGWH-X-LRVRLASHLRKLRKRLLR- | 11242 | 11242.9 | 57 |
| | X-C-PMO | | | |
| Myr-ApoE(133-150)-PMO | Myr-LRVRLASHLRKLRKRLLR-X-C-PMO | 9616.6 | 9617.1 | 55 |
| Myr-HA2-ApOE(133-150)-PMO | Myr-GLFHAIAHFIHGGWH-X-LRVRLASHLRKLRKRLLR- | 11410.3 | 11411.2 | 38 |
| | X-C-PMO | | | |
| Cy5-ApoE(141-150)-PMO | Cy5-LRKLRKRLLR-X-C-PMO | 9167.3 | 9168.5 | 64 |
| Cy5-HA2-ApoE(141-150)-PMO | Cy5-GLFHAIAHFIHGGWH-X-LRKLRKRLLR-X-C-PMO | 10962.2 | 10963.8 | 48 |
| Cy5-ApoE(133-150)-PMO | Cy5-LRVRLASHLRKLRKRLLR-X-C-PMO | 10100.4 | 10102 | 55 |
| Cy5-HA2-ApoE(133-150)-PMO | Cy5-GLFHAIAHFIHGGWH-X-LRVRLASHLRKLRKRLLR- | 11895.3 | 11896.6 | 35 |
| | X-C-PMO | | | |

Ac = acetylated; Myr = myristoylated; X = aminohexanoic acid; C = cysteine; PMO = antisense oligonucleotide
of SEQ ID NO:9

### Example 2—In Vitro Cell Uptake and Antisense Activity

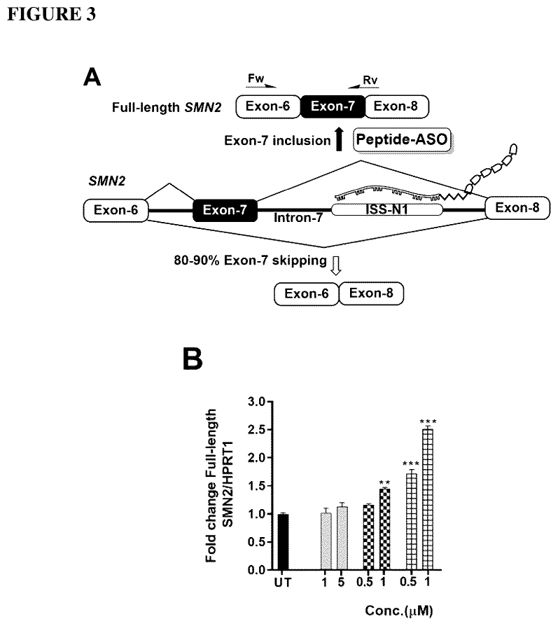

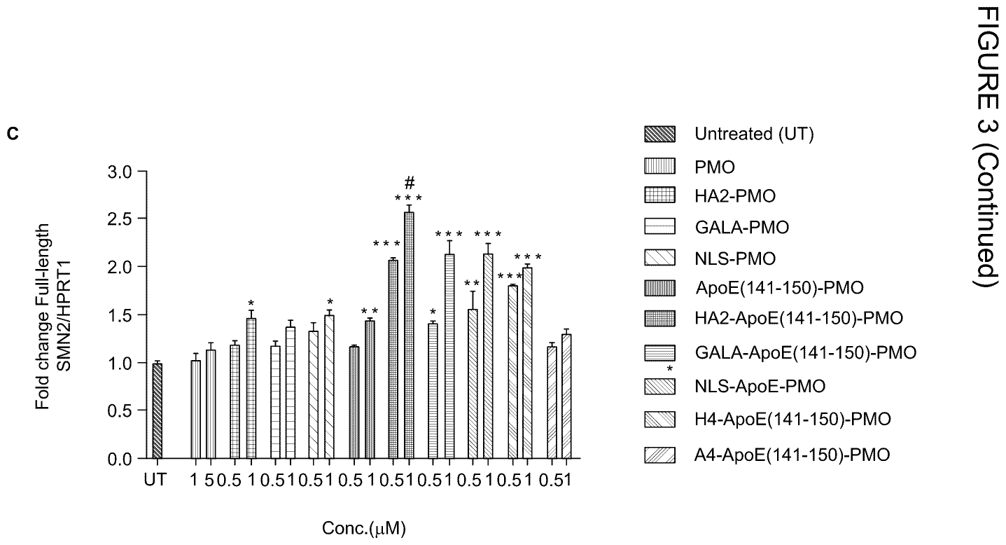

The inventors then evaluated cellular uptake and activity of these peptide-PMO conjugates in SMA Type I patient-derived fibroblasts with two copies of SMN2. Efficiency of uptake was assessed by RT-qPCR using SMN2 splice-switching for exon 7 inclusion (see FIG. 3A).

SMA Type I patient-derived fibroblasts (SMN2^{+/+}, GM03813, Coriell Cell Repositories) were maintained in Dulbecco's Modified Eagle's Medium (DMEM), supplemented with 10% foetal bovine serum (FBS), 1% Penicillin/Streptomycin and 1% L-glutamine (L-glu) at 37° C. Cells were plated in 6-well plates at a density of 3×10⁵/well in 6 well plates one day prior to treatment with PMO and peptide-PMO conjugates. Cells used were from passage 8-22. PMO and the peptide-PMO conjugates were dissolved in nuclease-free water and diluted to appropriate concentrations in Opti-MEM reduced serum medium. Cells were treated for 4 hrs at 37° C. The transfection medium was then removed and replaced with growth medium in which the cells were incubated for 20 hrs.

Cells were washed with PBS and total cellular RNA was extracted using Trizol (TRI Reagent®, Sigma) according to the manufacturer's instructions (Life Technologies). 400 ng purified RNA was reverse transcribed into cDNA using BioRad iScript Reverse Transcription Supermix kit according to the manufacturer's instructions. The reverse transcription was performed using the Veriti Thermal Cycler (ThermoFisher Scientific) with the thermal cycling conditions: 25° C. for 5 min, 46° C. for 20 min, 95° C. for 1 min followed by a 4° C. holding period.

Quantitative PCR (20 ng cDNA/well) was carried out in a 96-well plate in triplicate using SsoAdvanced Universal SYBR Green Supermix on a Biorad CFX96 Touch™ Real-Time PCR detection system. The amplification was carried out using full-length SMN2-specific primers: Ex-6 Forward 5′-GCTTTGGGAAGTATGTTAATTTCA-3′ (SEQ ID NO:10) and Ex7-8 reverse 5′CCTATGCCAGCATTTCTCCTTAATT-3′ (SEQ ID NO:11). Human HPRT1 was used as the internal reference gene with forward primer sequence 5′-GACCAGTCAACAGGGGACAT-3′ (SEQ ID NO:12) and reverse primer sequence 5′-CCTGACCAAGGAAAGCAAAG-3′ (SEQ ID NO:13). The ΔΔCt method was used to correct the Ct values against the HPRT1 Ct values. The values obtained were normalised to the untreated control values which were set to 1. Statistical analysis was carried out using one-way ANOVA with post-hoc Bonferroni for multiple groups comparison (GraphPad Prism V.8, 2018). All data are expressed as mean±SEM from at least three independent experiments with p<0.05 considered significant.

As shown in FIG. 3B, the level of exon-7 inclusion activity showed a concentration-dependent increase in activity for both ApoE(141-150)-PMO and ApoE(133-150)-PMO. ApoE(133-150)-PMO at 1 μM showed the highest level of exon-7 inclusion activity (2.51±0.06).

In order to investigate the effect of endosomal escape on the activity of the peptide-PMO conjugates, ApoE(141-150) was selected as it provides a better peptide model to show the effect of incorporation of endosomal escape domain. Endosomal escape peptides HA2, GALA and NLS were conjugated to PMO alone or as an N-terminal domain of ApoE(141-150). All showed a concentration-dependent increase in the exon-7 inclusion activity (FIG. 3C). Despite the low level of activity exhibited by the endosomal escape peptides conjugated to PMO alone, they imparted a significant increase in the activity of ApoE(141-150) sequence; the three conjugates, HA2-ApoE(141-150)-PMO, GALA-ApoE(141-150)-PMO and NLS-ApoE(141-150)-PMO showed a significant increase in the activity at both 0.5 μM and 1 μM. Amongst the endosomal escape domains employed, HA2 showed the highest efficiency. The activity of HA2-ApoE(133-150)-PMO conjugate was significantly higher at 0.5 μM (2.02±0.12) and 1 μM (2.8±0.13) compared to ApoE(133-150)-PMO at 0.5 μM (1.72±0.07) and 1 μM (2.51±0.06). The GALA-ApoE(141-150)-PMO also significantly increased the level of exon-7 inclusion at both 0.5 μM (1.41±0.04) and 1 μM (2.14±0.14) compared to GALA-PMO which showed no significant increase in the level of exon-7 inclusion activity. The addition of 4 histidine residues at the N-terminus of ApoE(141-150) resulted in a significant increase in the level of exon-7 inclusion of H4-ApoE(141-150)-PMO at 0.5 μM (1.81±0.02) and 1 μM (2.0±0.05) compared to ApoE(141-150)-PMO conjugate (FIG. 2C). Demonstrating that this enhanced activity was due to the histidine residues, replacement of the four histidine residues in H4-ApoE(141-150)-PMO with four alanine residues (A4-ApoE(141-150)-PMO) resulted in a significant decrease in the level of exon-7 inclusion activity.

The inventors have also shown that conjugation of myristic acid (Myr) at the N-terminus of the peptide-PMO conjugate ApoE(141-150)-PMO significantly increased activity (1.91±0.03) compared to its acetylated counterpart.

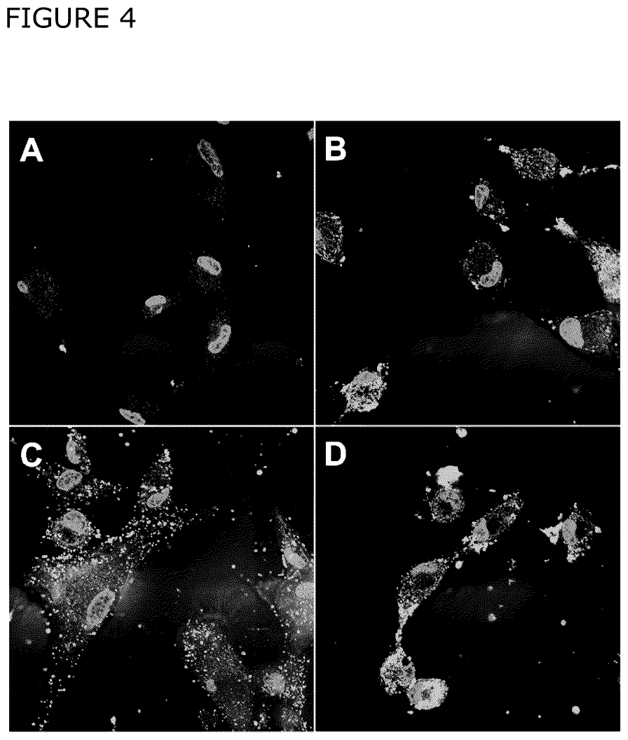

In order to assess the effect of endosomal escape peptide on the uptake efficiency of peptide-PMO conjugates, the most active peptide-PMO conjugates, Ac-HA2-ApoE(141-150)-PMO and Ac-HA2-ApoE(133-150)-PMO and their counterparts without endosomal escape domain HA2 (ApoE(141-150)-PMO and ApoE(133-150)-PMO), were labelled with Cy5 and fluorescence uptake was assessed. SMA fibroblasts (6.0×10⁴ cells/well) were plated onto a p-Slide 4 Well Ph+ Glass Bottom chamber slides precoated with poly-L-ornithine (Ibidi GmbH). After 24 hr, cells were incubated for 1 hr with Cy5-labelled peptide-PMO conjugates (1 μM or 5 μM) diluted in serum free Opti-MEM. At 10 min to end of 1 hr incubation, Hoechst (1:1000 dilution) was added to each well. Cells were washed with PBS and imaged using the Leica SP8 Resonant Scanning Confocal microscopy with 63×/1.4 oil objective The confocal imaging analysis of the fibroblasts treated with 1 μM of the Cy5-peptide-PMO conjugates showed that addition of endosomal escape domain HA2 significantly enhanced cellular uptake (FIG. 4).

The ApoE(141-150)-PMO and ApoE(133-150)-PMO conjugates showed a punctate intracellular fluorescence pattern (FIGS. 4A and 4C) indicative of the endosomal entrapment of the conjugate. This observation is consistent with previous studies which have shown endosomal sequestration results in a distinct punctate fluorescence signal (Fuchs and Raines, 2004, Biochemistry 43:2438-2444). However, the addition of HA2 resulted in a higher fluorescence intensity with a more diffused pattern, which reflects the endosomal release of the HA2-ApoE(141-150)-PMO (FIG. 4B) and HA2-ApoE(133-150)-PMO (FIG. 4D) conjugates. These results are in agreement with the exon-7 inclusion activity for these P-PMO conjugates (FIG. 3).

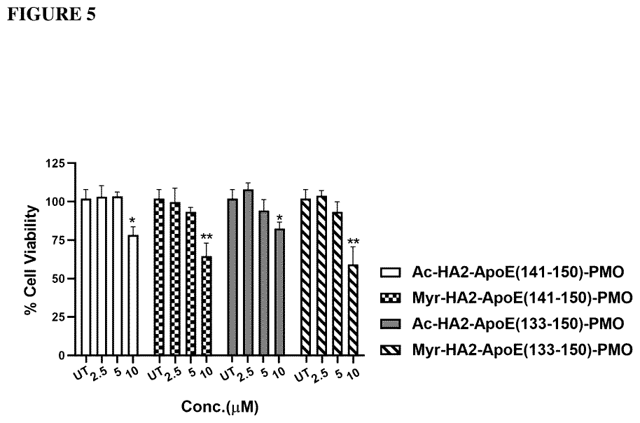

As a measure of cytotoxicity, the most active P-PMO conjugates with HA2 domain (HA2-ApoE(141-150)-PMO and HA2-ApoE(133-150)-PMO) and their myristoylated counterparts (Myr-HA2-ApoE(141-150)-PMO and Myr-HA2-ApoE(133-150)-PMO) were tested for effects on cell viability. The peptide-PMO conjugates were tested up to 10 μM which is 10-fold greater than the concentration needed to achieve a high level of activity. None of the P-PMO conjugates had a significant effect on cell viability up to 5 μM (FIG. 5). A decrease in cell viability was only observed at 10 μM.

### Example 3—In Vivo BBB Permeability: Increase in Full Length SMN2 Upon Administration of Ac-HA2-ApoE(133-150)-PMO

Based on the results described in Example 2, the inventors selected the most active peptide-PMO conjugate (Ac-HA2-ApoE(133-150)-PMO), based on generation of full length SMN2 levels, for in vivo analysis of CNS uptake and activity in transgenic SMN2^{+/-} mice.

A peptide consisting of the amino acid sequence of SEQ ID NO:6 was conjugated to the PMO antisense oligonucleotide of SEQ ID NO:9 via a non-reducible thioether (thiol-maleimide) bond as described in Example 1. As an initial step a dose-escalation study was carried out at 5, 8 and 10 mg/kg, and the 8 mg/kg was determined to be well-tolerated without any signs of adverse effects (data not shown). Transgenic SOD1^{+/-} SMN2^{+/-} mice (n=5) at P60 were treated with Ac-HA2-ApoE(133-150)-PMO conjugate at 8 mg/kg/week for 5 weeks via tail vein injection. Age-matched control mice (n=6) were given saline. Tissues (brain, spinal cord, kidney and quadriceps) were harvested 7 days after final administration. The level of full-length SMN2 RNA was measured using RT-qPCR as above. The level of full-length SMN2 was normalised to mouse HPRT1 with forward primer sequence 5′-GATCAGTCAACGGGGGACAT-3′ (SEQ ID NO:14) and reverse primer sequence 5′CCATTTTGGGGCTGTACTGCTT-3′ (SEQ ID NO:15). Data were expressed as mean±SEM. The statistical significances between saline-treated and Ac-HA2-ApoE(133-150)-PMO-treated groups were determined using the Student's t test. A P value of <0.05 was considered statistically significant.

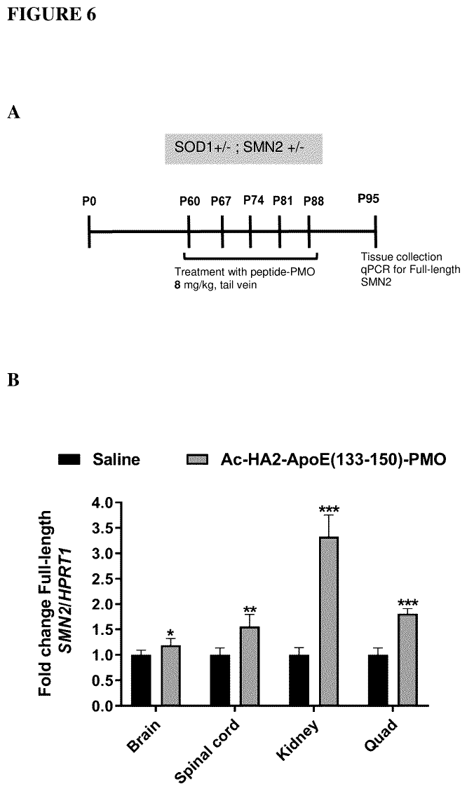

As shown in FIG. 6B, administration of Ac-HA2-ApoE(133-150)-PMO to transgenic SOD1 mice (SOD1^{+/-}; SMN2^{+/-}) resulted in a significant increase in the level of full length SMN2 RNA in the brain (1.19±0.06) and spinal cord (1.56±0.1) compared to the saline-treated control.

### Example 4—Fusion Peptides Comprising TPA Functional Group

The inventors have developed a series of fusion peptides, based on the HA2-ApoE(133-150) and ApoE(141-150) peptides described above, comprising the functional group thiopropionic acid (TPA) either at the N-terminus or between the HA2 domain and ApoE(133-150) as shown in Table 3. The inclusion of TPA allows for an active agent, such as an antisense oligonucleotide, to be conjugated to the TPA as a branched or linear construct (see FIG. 1C). The peptides were constructed using an amino-PEG2-acetic acid (miniPEG) spacer between the HA2 and ApoE domains and/or between the TPA and the HA2 domain (see Table 3), to provide flexibility to the construct and enable each of the HA2, ApoE and the active agent to better interact with their respective targets for higher cell uptake and activity.

In an effort to increase stability of the fusion peptides TPA-ApoE(133-150), TPA-HA2-ApoE(141-150), branched HA2-(TPA)-ApoE(141-150), TPA-HA2-ApoE(133-150) and branched HA2-(TPA)-ApoE(133-150) shown in Table 3 without compromising receptor binding affinity, the inventors also constructed retro-inverso equivalents of these peptides in which the peptide sequences were synthesised in reverse order using D-amino acids (see RI peptides in Table 3). This ensures the side chains of the amino acids in the sequence have the same spatial orientation as in the L-peptide, for interaction with receptors.

Serum stability analysis was conducted for each peptide (7×50 nmol) shown in Table 3 in 50% human serum (100 μL) at 37° C. At the end of each time point (0, 10, 30, 60, 120, 240, and 480 min), 1 M guanidine hydrochloride solution (200 μL) and ice-cold acetonitrile (600 μL) were added, and the serum-peptide solution was centrifuged at 14000 g for 10 min. The supernatant was lyophilised and redissolved in water (200 μL). All samples were analysed by RP-HPLC in the presence of phenol (50 nmol) as an internal standard, using water and acetonitrile containing 0.1% TFA, with a gradient of 10-80% acetonitrile over 30 min, and a flow rate of 1.5 mL/min. The percentage of peptide remaining was measured based on the area under curve. The percentage of peptide remaining versus time data was fitted to a non-linear one-phase exponential decay model in GraphPad Prism 9.2.0 to determine the half-life for each peptide.

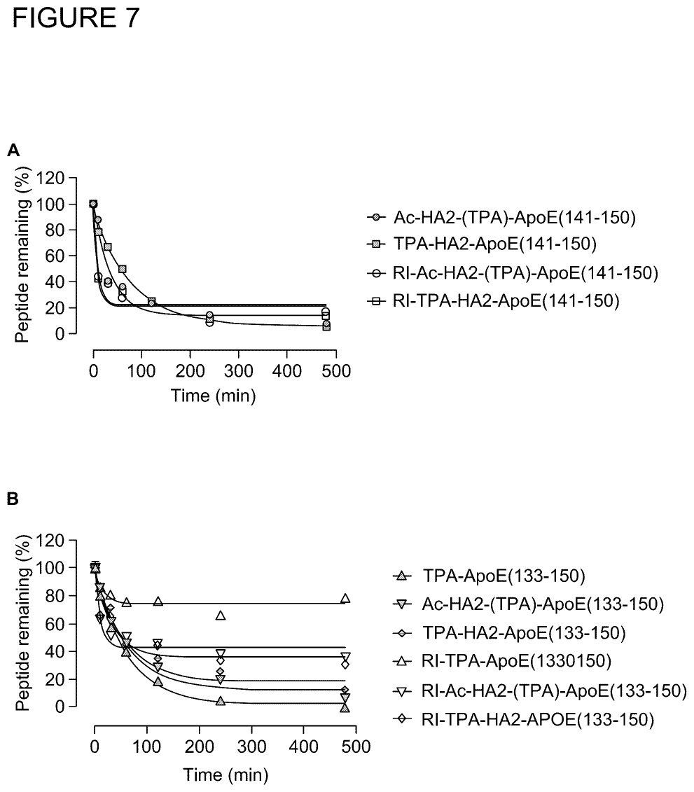

As shown in Table 3 and FIG. 7, the retro-inverso peptides RI TPA-ApoE(133-150), RI TPA-HA2-ApoE(133-150) and RI Br-HA2-(TPA)-ApoE(133-150) showed significantly higher proteolytic stability compared to the parent L-peptides TPA-ApoE(133-150), TPA-HA2-ApoE(133-150) and Br-HA2-(TPA)-ApoE(133-150).

TABLE 3

| | | |
|---|---|---|
| | | Serum |
| | | Stability |
| Peptide | Sequence | (h) |

Peptides containing natural L-amino acids

| | | |
|---|---|---|
| TPA-HA2-ApoE(141-150) | TPA-mPEG-GLFHAIAHFIHGGWH-mPEG-LRKLRKRLLR | 1 |
| Br-HA2-(TPA)-ApoE(141-150) | Ac-GLFHAIAHFIHGGWH-mPEG-K(mPEG-TPA)-mPEG-LRKLRKRLLR | 0.6 |
| TPA-ApoE(133-150) | TPA-mPEG-LRVRLASHLRKLRKRLLR | 0.73 |
| TPA-HA2-ApoE(133-150) | TPA-mPEG-GLFHAIAHFIHGGWH-mPEG-LR VRLASHLRKLRKRLLR | 1.1 |
| Br-HA2-(TPA)-ApoE(133-150) | Ac-GLFHAIAHFIHGGWH-mPEG-K(mPEG-TPA)-mPEG-LRVRLASHLR | 1 |
| | KLRKRLLR | |

Retroinverso (RI) peptides: peptides synthesised in reverse order using D-amino acids

| | | |
|---|---|---|
| RI TPA-HA2-ApoE(141-150) | TPA-mPEG-hwgghifhaiahflg-mPEG-K-mPEG-rllrkrlkrl | 0.4 |
| RI Br-HA2-(TPA)-ApoE(141-150) | Ac-hwgghifhaiahflg-mPEG-K(mPEG-TPA)-mPEG-rllrkrlkrl | 0.3 |
| RI TPA-ApoE(133-150) | TPA-mPEG-rllrkrlkrlhsalrvrl | 11.2 |
| RI TPA-HA2-ApoE(133-150) | TPA-mPEG-hwgghifhaiahflg-mPEG-K-mPEG-rllrkrlkrlhsal | 1.6 |
| | rvrl | |
| RI Br-HA2-(TPA)-ApoE(133-150) | Ac-hwgghifhaiahflg-mPEG-K(mPEG-TPA)-mPEG-rllrkrlkrl | 1.6 |
| | hsalrvrl | |

Br = branched; RI = retroinverso; TPA = thiopropionic acid; mPEG = miniPeg spacer
Branched refers to the branched nature of a conjugate when an active agent is conjugated to the fusion peptide (e.g. as illustrated in Figure 1C).

### Example 5—Efficiency of Cellular Uptake of TPA-Containing Fusion Peptides Conjugated with PMO

The inventors constructed peptide-PMO conjugates of the peptides described in Table 3 and the PMO oligonucleotide described in Example 1 according to the methods described in Example 1. Cellular uptake efficiency of the resulting peptide-PMO conjugates and the ability of the conjugates to upregulate the level of full length SMN2 in SMA patient-derived fibroblasts was assessed by RT-qPCR using SMN2 splice-switching for exon-7 inclusion.

SMA Type-I patient-derived fibroblasts (GM03813, Coriell Cell Re-positories) were maintained in Dulbecco's Modified Eagle's Medium (DMEM), supplemented with 10% foetal bovine serum, 1% penicillin/streptomycin and 1% L-glutamine at 37° C. Cells were plated at a density of 5×10⁵ cells/well in 6-well plates one day prior to treatment with PMO and peptide-PMO conjugates. Peptide-PMO conjugates were dissolved in nuclease-free water and diluted to appropriate concentrations (0.25, 0.5, and 1 μM) in Opti-MEM reduced serum medium. The SMA fibroblasts were treated for 4 hr. The transfection medium was removed and replaced with growth medium and incubated for further 20 hr.

Total RNA was extracted from cells and using Bioline ISOLATE II RNA mini kit according to the manufacturer's instructions (Meridian Bioscience). Using a BioRad iScript Reverse Transcription Supermix kit, 400 ng of purified RNA was reverse transcribed to single-stranded complementary DNA (cDNA). The transcription was performed using the Veriti Thermal Cycler (ThermoFisher Scientific) with the thermal cycling conditions: 25° C. for 5 min, 46° C. for 20 min, 95° C. for 1 min followed by a 4° C. holding period. Quantitative PCR was subsequently carried out using 20 ng of cDNA per well of 96-well plate in triplicates for each treatment, using SsoAdvanced Universal SYBR Green Supermix on a Biorad CFX96 Real-Time PCR detection system. The amplifications were carried out under the following thermal conditions: 95° C. for 2 min, followed by 39 cycles of amplifications, with 95° C. for 5 s and 60° C. for 30 s, then 95° C. for 5 s. Full length SMN transcripts were amplified using a forward primer in exon 6 (5′-GCTTTGGGAAGTATGTTAATTTCA-3′; SEQ ID NO:10) and a reverse primer spanning exons 7-8 (5′-CTATGCCAGCATTTCTCCTTAATT-3′; SEQ ID NO:11). Human HPRT1 was used as the internal reference gene with forward primer sequence 5′-GACCAGTCAACAGGGGACAT-3′ (SEQ ID NO:12) and reverse primer sequence 5′CCCTGACCAAGGAAAGCAAAG-3′ (SEQ ID NO:13). The ΔΔCt method was used to correct the Ct values against the HPRT1 Ct values. The values obtained were normalised to the untreated control values which were set to 1.

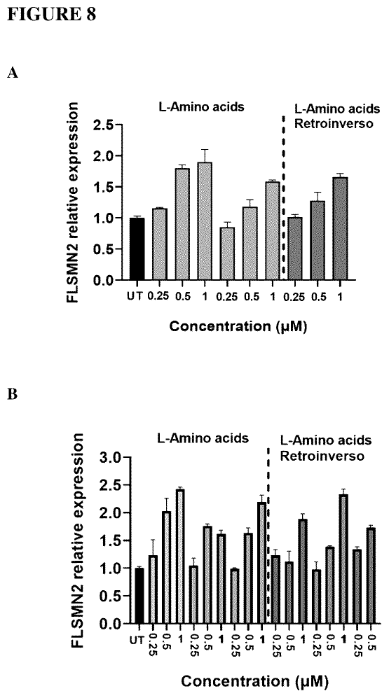

As shown in FIG. 8, all peptide-PMO conjugates significantly increased the level of full-length SMN2 in a concentration-dependent manner. The HA2-ApoE(141-150)-PMO conjugate in a linear format (FIG. 8A) showed a slightly higher activity compared to its branched counterpart (Br-HA2-ApoE(141-150). There was no difference in the activity of the branched ApoE(141-150)-PMO and its retro-inverso version (RI-Br-ApoE(141-150)-PMO). This suggests that synthesis of the branched ApoE(141-150) peptide in reverse order with D-amino acids instead of L-amino acids has no effect on cellular uptake and activity.

A similar pattern of concentration-dependent increase in activity was observed for ApoE(133-150)-PMO and HA2-ApoE (133-150)-PMO conjugates peptide sequences (FIG. 8B). The activity of the linear and branched versions were comparable and there was no difference in the activity of the L-peptides and the retro-inverso D-peptides.

1. A fusion peptide comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain.

2. A fusion peptide according to claim 1, wherein the fragment comprises the amino acid sequence of SEQ ID NO:2 or SEQ ID NO:25 or a or derivative thereof, or a sequence at least about 80% identical thereto.

3. A fusion peptide according to claim 1, wherein the peptide sequence of (i) comprises or consists of the amino acid sequence of SEQ ID NO:1 or SEQ ID NO:24 or a derivative thereof or a sequence at least about 80% identical thereto.

4. A fusion peptide according to claim 1, wherein the peptide sequence of (i) comprises or consists of the amino acid sequence of SEQ ID NO:2 or SEQ ID NO:25 or a derivative thereof or a sequence at least about 80% identical thereto.

5. A fusion peptide according to any one of claims 1 to 4, wherein the peptide sequence of (i) is linked to the endosomal escape domain via a linker.

6. A fusion peptide according to claim 5, wherein the endosomal escape domain comprises an N-terminal fragment of the influenza virus hemagglutinin subunit HA2, a GALA peptide or a nuclear localisation sequence.

7. A fusion peptide according to claim 6, wherein the fragment of HA2 comprises the amino acid sequence of SEQ ID NO:3 or SEQ ID NO:26, a derivative thereof, or a sequence at least about 80% identical thereto.

8. A fusion peptide according to any one of claims 1 to 7, wherein the endosomal escape domain is linked to the N-terminal end of the peptide sequence of (i).

9. A fusion peptide according to any one of claims 1 to 8, wherein the fusion peptide comprises a lipid moiety linked to the N-terminal end.

10. A fusion peptide according to claim 9, wherein the lipid moiety comprises a myristoyl group.

11. A fusion peptide according to any one of claims 1 to 10, wherein the fusion peptide further comprises an active agent conjugated thereto.

12. A fusion peptide comprising (i) a peptide sequence comprising the amino acid sequence of SEQ ID NO:1, a derivative thereof, or a sequence at least about 80% identical thereto and (ii) a peptide sequence comprising the amino acid sequence of SEQ ID NO:3, a derivative thereof, or a sequence at least about 80% identical thereto.

13. Use of a fusion peptide comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain, for the targeted delivery of an active agent to the central nervous system (CNS) across the blood-brain barrier.

14. Use of a fusion peptide comprising (i) a peptide sequence comprising the amino acid sequence of SEQ ID NO:1 or SEQ ID NO:24 or a fragment or derivative thereof, or a sequence at least about 80% identical thereto and (ii) an endosomal escape domain, for the targeted delivery of an active agent to the eye across a blood-ocular barrier.

15. Use according to claim 14, wherein the blood-ocular barrier is the blood-aqueous barrier or the blood-retinal barrier.

16. Use according to any one of claims 13 to 15, wherein the active agent is linked to component (i) or component (ii) of the fusion peptide.

17. Use according to claim 16, wherein the active agent is linked to the fusion peptide via a linker.

18. Use according to any one of claims 13 to 17, wherein the active agent is a therapeutic agent, a diagnostic agent or an imaging agent.

19. A method for delivering an active agent across the blood-brain barrier to the CNS, comprising linking the active agent to a fusion peptide according to any one of claims 1 to 12 and systemically administering the resultant compound to a subject in need thereof, wherein said administration results in delivery of the active agent across the blood-brain barrier to the CNS.

20. A method for delivering an active agent across a blood-ocular barrier to the eye, comprising linking the active agent to a fusion peptide according to any one of claims 1 to 12 and systemically administering the resultant compound to a subject in need thereof, wherein said administration results in delivery of the active agent across a blood-ocular barrier to the eye.

21. A method according to claim 19 or 20, wherein the active agent is a therapeutic agent, a diagnostic agent or detection agent.

22. A method according to any one of claims 19 to 21, wherein the active agent is a therapeutic agent for the treatment of an ocular disorder, a neurological disorder, a neuromuscular disorder, or other disorder affecting the CNS.

23. A method according to any one of claims 19 to 21, wherein the active agent is a diagnostic agent for the detection and diagnosis of an ocular disorder, a neurological disorder, a neuromuscular disorder, or other disorder affecting the CNS.

24. A method according to any one of claims 19 to 21, wherein the active agent is a detection agent for detecting, and optionally imaging, biological tissue within the central nervous system or eye and/or abnormal structures of the central nervous system or eye.

25. A method for increasing the bioavailability in the CNS or eye of an active agent, comprising linking the active agent to a fusion peptide according to any one of claims 1 to 12 and systemically administering the resultant compound to a subject in need thereof, wherein said administration results in an increased bioavailability of the active agent in the CNS or eye, compared to the delivery of the active agent in the absence of the fusion peptide.

26. A method for treating or preventing a disorder of the CNS, or at least one symptom thereof, in a subject, comprising systemically administering to the subject an effective amount of therapeutic agent linked to a fusion peptide according to any one of claims 1 to 12, wherein the peptide conjugate facilitates delivery of the therapeutic agent across the blood-brain barrier to the CNS.

27. A method according to claim 26, wherein the disorder of the CNS is a neurological or neuromuscular disorder.

28. A method for diagnosing a disorder of the CNS in a subject, comprising systemically administering to the subject a diagnostic agent linked to a fusion peptide according to any one of claims 1 to 12, wherein the fusion peptide facilitates delivery of the diagnostic agent across the blood-brain barrier to the CNS.

29. A method for visualising a region or structure of the CNS, comprising systemically administering to the subject imaging agent comprising a CNS tissue-targeting moiety and a detectable label linked to an imaging moiety and a fusion peptide according to any one of claims 1 to 12, wherein the fusion peptide facilitates delivery of the detection agent across the blood-brain barrier to the CNS.

30. A method for treating or preventing an ocular disorder, or at least one symptom thereof, in a subject, comprising systemically administering to the subject an effective amount of therapeutic agent linked to a fusion peptide according to any one of claims 1 to 12, wherein the peptide conjugate facilitates delivery of the therapeutic agent across a blood-ocular barrier to the eye.

31. A method for diagnosing an ocular disorder in a subject, comprising systemically administering to the subject a diagnostic agent linked to a fusion peptide according to any one of claims 1 to 12, wherein the fusion peptide facilitates delivery of the diagnostic agent across a blood-ocular barrier to the eye.

32. A method for visualising a region or structure of the eye, comprising systemically administering to the subject imaging agent comprising an ocular tissue-targeting moiety and a detectable label linked to an imaging moiety and a fusion peptide according to any one of claims 1 to 12, wherein the fusion peptide facilitates delivery of the detection agent across a blood-ocular barrier to the eye.

33. A method according to any one of claims 30 to 32, wherein the blood-ocular barrier is the blood-aqueous barrier.

34. A method according to any one of claims 30 to 32, wherein the blood-ocular barrier is the blood-retinal barrier.


SEQUENCE LISTING (26 sequences)

SEQ ID NO: 1 - protein, 18 aa, Homo sapiens
LRVRLASHLRKLRKRLLR

SEQ ID NO: 2 - protein, 10 aa, Homo sapiens
LRKLRKRLLR

SEQ ID NO: 3 - protein, 15 aa, Homo sapiens
GLFHAIAHFIHGGWH

SEQ ID NO: 4 - protein, 30 aa, Artificial Sequence
  Synthetic sequence
WEAALAEALAEALAEHLAEALAEALEALAA

SEQ ID NO: 5 - protein, 10 aa, Artificial Sequence
  Synthetic sequence
VQRKRQKLMP

SEQ ID NO: 6 - protein, 34 aa, Artificial Sequence
  Synthetic sequence
  Linker (16)..(16)
GLFHAIAHFIHGGWHXLRVRLASHLRKLRKRLLR

SEQ ID NO: 7 - protein, 26 aa, Artificial Sequence
  Synthetic sequence
  Linker (16)..(16)
GLFHAIAHFIHGGWHXLRKLRKRLLR

SEQ ID NO: 8 - protein, 18 aa, Artificial Sequence
  Synthetic sequence
TCACTTTCATAATGCTGG

SEQ ID NO: 9 - DNA, 20 bp, Artificial Sequence
  Synthetic sequence
attcactttcataatgctgg

SEQ ID NO: 10 - DNA, 24 bp, Artificial Sequence
  Synthetic sequence
gctttgggaagtatgttaatttca

SEQ ID NO: 11 - DNA, 24 bp, Artificial Sequence
  Synthetic sequence
ctatgccagcatttctccttaatt

SEQ ID NO: 12 - DNA, 20 bp, Artificial Sequence
  Synthetic sequence
gaccagtcaacaggggacat

SEQ ID NO: 13 - DNA, 20 bp, Artificial Sequence
  Synthetic sequence
cctgaccaaggaaagcaaag

SEQ ID NO: 14 - DNA, 20 bp, Artificial Sequence
  Synthetic sequence
gatcagtcaacgggggacat

SEQ ID NO: 15 - DNA, 21 bp, Artificial Sequence
  Synthetic sequence
cattttggggctgtactgctt

SEQ ID NO: 16 - protein, 34 aa, Artificial Sequence
  Synthetic sequence
  Linker (1)..(1): Thiopropionic acid
  MISC_FEATURE (1)..(2): miniPEG spacer between positions 1 and 2
  MISC_FEATURE (16)..(17): miniPEG spacer between positions 16 and 17
XGLFHAIAHFIHGGWHLRVRLASHLRKLRKRLLR

SEQ ID NO: 17 - protein, 35 aa, Artificial Sequence
  Synthetic sequence
  MISC_FEATURE (15)..(16): miniPEG spacer between locations 15 and 16
  MISC_FEATURE (16)..(17): miniPEG spacer between locations 16 and 17
  Linker (17)..(17): Thiopropionic acid
  MISC_FEATURE (17)..(18): miniPEG spacer between locations 17 and 18
GLFHAIAHFIHGGWHKXLRVRLASHLRKLRKRLLR

SEQ ID NO: 18 - protein, 26 aa, Artificial Sequence
  Synthetic sequence
  Linker (1)..(1): Thiopropionic acid
  MISC_FEATURE (1)..(2): miniPEG spacer between positions 1 and 2
  MISC_FEATURE (16)..(17): miniPEG spacer between positions 16 and 17
XGLFHAIAHFIHGGWHLRKLRKRLLR

SEQ ID NO: 19 - protein, 27 aa, Artificial Sequence
  Synthetic sequence
  MISC_FEATURE (15)..(16): miniPEG spacer between positions 15 and 16
  MISC_FEATURE (16)..(17): miniPEG spacer between positions 16 and 17
  Linker (17)..(17): Thiopropionic acid
  MISC_FEATURE (17)..(18): miniPEG spacer between locations 17 and 18
GLFHAIAHFIHGGWHKXLRKLRKRLLR

SEQ ID NO: 20 - protein, 35 aa, Artificial Sequence
  Synthetic sequence
  Linker (1)..(1): Thiopropionic acid
  MISC_FEATURE (1)..(2): miniPEG spacer between positions 1 and 2
  MISC_FEATURE (16)..(17): miniPEG spacer between positions 16 and 17
  MISC_FEATURE (17)..(18): miniPEG spacer between positions 17 and 18
XHWGGHIFHAIAHFLGKRLLRKRLKRLHSALRVRL

SEQ ID NO: 21 - protein, 35 aa, Artificial Sequence
  Synthetic sequence
  MISC_FEATURE (15)..(16): miniPEG spacer between positions 15 and 16
  MISC_FEATURE (16)..(17): miniPEG spacer between positions 16 and 17
  Linker (17)..(17): Thiopropionic acid
  MISC_FEATURE (17)..(18): miniPEG spacer between positions 17 and 18
HWGGHIFHAIAHFLGKXRLLRKRLKRLHSALRVRL

SEQ ID NO: 22 - protein, 27 aa, Artificial Sequence
  Synthetic sequence
  Linker (1)..(1): Thiopropionic acid
  MISC_FEATURE (1)..(2): miniPEG spacer between positions 1 and 2
  MISC_FEATURE (16)..(17): miniPEG spacer between positions 16 and 17
  MISC_FEATURE (17)..(18): miniPEG spacer between positions 17 and 18
XHWGGHIFHAIAHFLGKRLLRKRLKRL

SEQ ID NO: 23 - protein, 27 aa, Artificial Sequence
  Synthetic sequence
  MISC_FEATURE (15)..(16): miniPEG spacer between positions 15 and 16
  MISC_FEATURE (16)..(17): miniPEG spacer between positions 16 and 17
  Linker (17)..(17): Thiopropionic acid
  MISC_FEATURE (17)..(18): miniPEG spacer between positions 17 and 18
HWGGHIFHAIAHFLGKXRLLRKRLKRL

SEQ ID NO: 24 - protein, 18 aa, Artificial Sequence
  Synthetic sequence
RLLRKRLKRLHSALRVRL

SEQ ID NO: 25 - protein, 10 aa, Artificial Sequence
  Synthetic sequence
RLLRKRLKRL

SEQ ID NO: 26 - protein, 15 aa, Artificial Sequence
  Synthetic sequence
HWGGHIFHAIAHFLG